(19) United States
(12) Patent Application Publication
(10) Pub. No.: US 2017/0208834 A1
(43) Pub. Date: Jul. 27, 2017

(54) PROTEIN CONTAINING MATERIAL FROM BIOMASS AND METHODS OF PRODUCTION

(71) Applicant: SYNTHETIC GENOMICS, INC., La Jolla, CA (US)

(72) Inventors: Johannes Scholten, San Diego, CA (US); Arun Lakshmanaswamy, San Diego, CA (US); Joel Burke, San Diego, CA (US); George C. Rutt, San Diego, CA (US)

(21) Appl. No.: 15/417,132

(22) Filed: Jan. 26, 2017

Related U.S. Application Data
(60) Provisional application No. 62/287,837, filed on Jan. 27, 2016.

(51) Int. Cl. A23J 1/00; A23J 3/20; C12P 21/00; C07K 1/14; C07K 14/405

(52) U.S. Cl. CPC A23J 1/009; C07K 1/145; C07K 14/405; C12P 21/00; A23J 3/20

(57) ABSTRACT

The present invention provides methods and protein compositions having advantageous properties, such as a high uncorrected limiting amino acid score as well as favorable amounts of essential amino acids, branched chain amino acids, as well as other amino acids more difficult to find in the regular diet. The protein composition is obtainable as taught herein from algal or microbial biomass. The protein composition produced according to the methods of the invention provides a proteinaceous food or food ingredient that is more nutritionally balanced (and therefore nutritionally superior) to protein compositions otherwise available. The protein material is advantageously used as a food or food ingredient for humans and/or animals. Also provided are methods of producing the protein material from biomass sources.

### CROSS-REFERENCE TO RELATED APPLICATIONS

This application claims benefit of priority under 35 U.S.C. §119(e) of U.S. Patent Application Ser. No. 62/287,837, filed Jan. 27, 2016, the entire contents of which is incorporated herein by reference in its entirety. This application also incorporates by reference in its entirety U.S. patent application Ser. No. 15/005,695, filed Jan. 25, 2016, including all tables, figures, and claims.

### FIELD OF THE INVENTION

The present invention relates to protein containing material derived from biomass and methods of producing same.

### BACKGROUND OF THE INVENTION

Proteins are essential nutritional components and protein rich material is often added to various types of food products in order to increase the nutritional content. Current sources of protein material include various grains and animal sources, but their availability is often subject to wide seasonal fluctuations, limiting their commercial use by food manufacturers. Grain-based solutions for protein production also consume a large amount of productive land and water resources that might otherwise be better utilized. These sources are also limited in their ability to supply sustainable supplies of proteins in the quantities necessary. Additional and more reliable sources of proteins are needed to supply both a growing humanity and as feed for domesticated animals.

Algal and microbial sources of proteins or other nutritional materials have great potential and would be highly desirable as they can reduce seasonal fluctuations and nevertheless provide a consistent, economic, and sustainable source of nutritional materials to food providers. Proteins and other nutritional materials produced by these sources could be used to supplement cereals, snack bars, and a wide variety of other food products. Furthermore, if organisms dependent on photosynthesis for energy (e.g., algae) could be made to produce useable proteins, it would have a highly favorable effect on the energy equation in food production.

However, algal and microbial sources of proteins often suffer from significant disadvantages in that they contain substances that are severely displeasing in terms of their organoleptic taste and smell properties. These sources of proteins also have disadvantages shared with other protein sources, which is that the content of the proteins they contain is not optimally balanced for human or animal nutritional needs. The may further contain allergens that are harmful to some people and be nutritionally deficient in having amino acids that are out of balance for human and animal needs.

It would be highly advantageous to be able to harvest proteins from algal and microbial organisms that do not have the displeasing organoleptic properties and the other disadvantages and to be able to harvest such proteins in a manner that yields proteins having a more balanced nutritional profile advantageous for human and animal needs. Such proteins would be very useful as foods, food ingredients, and nutritional supplements.

### SUMMARY OF THE INVENTION

The present invention provides methods and protein compositions having advantageous properties, such as a high uncorrected limiting amino acid score as well as favorable amounts of essential amino acids, branched chain amino acids, as well as other amino acids more difficult to find in the regular diet. The protein composition is obtainable as taught herein from algal or microbial biomass. The protein composition obtainable according to the methods of the invention provides a proteinaceous food or food ingredient that is more nutritionally balanced (and therefore nutritionally superior) to protein compositions otherwise available. The protein material is advantageously used as a food or food ingredient for humans and/or animals. Also provided are methods of isolating the protein material from biomass sources.

In a first aspect the invention provides a protein composition derived from cellular biomass and having an uncorrected limiting amino acid score of 0.88 or greater for all essential amino acids. The biomass can be derived from algae, for example heterotrophic algae. In some embodiments the protein composition has an uncorrected limiting amino acid score of greater than 0.94 for all essential amino acids, or greater than 1.0 for all essential amino acids. The protein composition can contain phe in an amount of 3.5% of total protein or greater, and tyr in an amount of 2.75% of total protein or greater.

In various embodiments the protein composition can have any one or more of a protein content of greater than 65%, a lipid content is less than 10% or less than 2%, and an ash content is less than 8%. The content of essential amino acids can be greater than 35% of total protein. The content of branched chain amino acids can be greater than 16% of total protein.

In some embodiments the protein composition can contain any one or more of a leucine in an amount greater than 5.5% of total protein; isoleucine in an amount greater than 3.0% of total protein; glutamic acid in an amount less than 20% of total protein; lysine in an amount greater than 5.5% of total protein; and/or valine in an amount greater than 4.5% of total protein. In another embodiment the composition can contain any one or more of leucine in an amount greater than 6% of total protein; lysine in an amount greater than 6% of total protein; and/or glutamic acid in an amount less than 15% of total protein.

The protein composition can have organoleptic taste and smell properties that are acceptable to a human, which can be at least equivalent to soy. In some embodiments the protein composition derived from heterotrophic algae of the class Labyrinthulomycetes, which in various embodiments can a Thraustochytrium, an Aurantiochytrium, or a Schizochytrium. The protein composition can be derived from a single source. In some embodiments the protein composition does not contain human allergens from any one or more of peanut, milk, soy, nut, egg, whey, wheat, fish, shellfish, or pea at or above the lowest observed adverse effect level for the particular human allergen.

In another aspect the invention provides a method of producing a protein composition described herein. The method can involve steps of cultivating a cellular biomass in a defined medium; delipidating the biomass; exposing the delipidated biomass to acidic conditions by adjusting the pH of the biomass to a depressed pH of less than 4.5 and holding the pH of the biomass at said depressed pH for at least 10 minutes; and harvesting a protein composition described herein. Exposing the delipidated biomass to acidic conditions can involve exposing the biomass to a pH of about 3.5 and the pH is held for about 30 minutes. The cellular biomass can be from algal biomass or any described herein.

### DESCRIPTION OF THE DRAWINGS

FIG. 1 provides the FAO recommended requirements for persons of various ages.

FIG. 2 is a graphical illustration comparing amino acid content (% amino acid/total amino acids) for biomass grown in a rich medium containing organic nitrogen versus a defined medium of Table 1.

FIG. 3 is a graphical illustration of the removal of lipidic material at steps of a process of the invention.

FIG. 4 is a flow chart showing steps that can be used in various embodiments of the methods of the invention. Not all steps need be included in every embodiment of the methods. The steps can be performed in the order shown in FIG. 4, or in a different order.

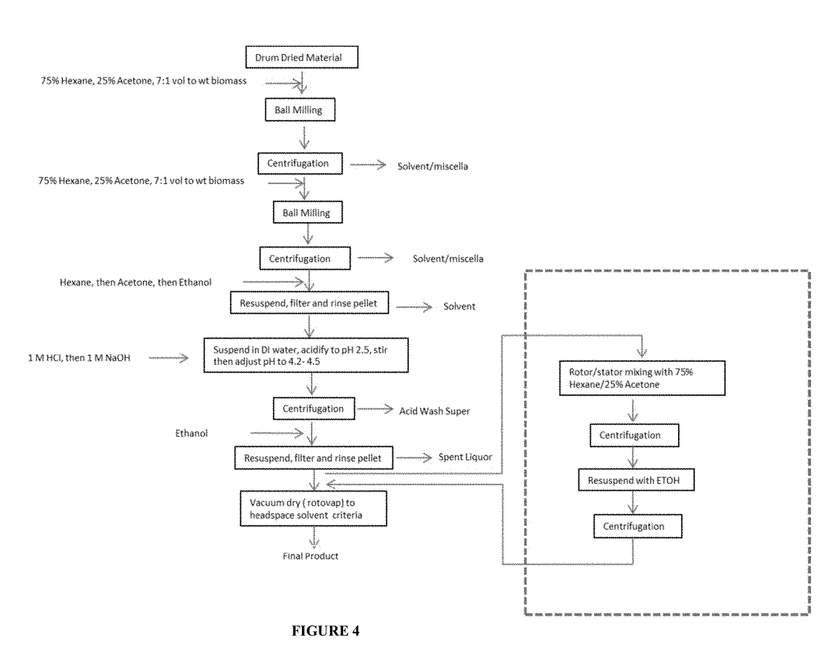

### DETAILED DESCRIPTION OF THE INVENTION

The present invention provides a composition containing a protein material useful as a food or food ingredient or food supplement or food substitute for humans and/or animals. The protein material can be derived from biomass and has advantageous properties, such as any one or more of an advantageous nutritional profile in terms of the amino acid content, branched-chain amino acid content, essential amino acid content, phenylalanine and tyrosine content, arginine and glutamic acid/glutamine content, and methionine and cysteine content of the protein. The nutritional profile of the protein material of the invention can also have an advantageous level of overall protein content and/or low ash content and/or desirable fat, carbohydrate, and moisture content. In various embodiments the protein material has an uncorrected limiting amino acid (UCLAA) score of greater than 0.68 or greater than 0.70 or greater than 0.72 or greater than 0.74 or greater than 0.76 or greater than 0.78 or greater than 0.80 or greater than 0.82 or greater than 0.84 or greater than 0.86 or greater than 0.87 or greater than 0.88 or greater than 0.89 or greater than 0.90 or greater than 0.91 or greater than 0.92 or greater than 0.93 or greater than 0.94 or greater than 0.95 or greater than 0.96 or greater than 0.97 or greater than 0.98 or greater than 0.99 or greater than 1.00 or greater than 1.01 or greater than 1.03 or greater than 1.05 or greater than 1.07 for all essential amino acids. In some embodiments the UCLAA score for any one or more or all essential amino acids is at least 5% higher or at least 7% or at least 10% or at least 12% or at least 14% or at least 15% or at least 18% or at least 20% or at least 22% or at least 24% higher when the biomass organisms are grown in a defined medium as disclosed herein versus a rich medium. This is very advantageous because most protein sources from biomass sources have a UCLAA score of less than 0.90 or less than 0.86.

Amino acid scoring can be used to measure how efficiently a protein will meet the nutritional needs of a person (or animal). It can also be used as an uncorrected measure of the amino acid content of a particular protein. In the present case the uncorrected limiting amino acid (UCLAA) score is a measure of the amino acid content of a particular protein material. The amino acids that are included in the essential amino acids may vary depending on the animal consumer of the protein composition. The nine essential amino acids for humans are histidine, isoleucine, leucine, lysine, methionine, phenylalanine, threonine, tryptophan, and valine. Consistent with practice in the art the amount of met in a protein material can be measured in combination with cysteine as met+cys, and the amount of phe can be measured in combination with tyrosine as phe+tyr. Thus, in some embodiments the protein compositions of the invention have a UCLAA score of greater than 0.68 or greater than 0.70 or greater than 0.72 or greater than 0.74 or greater than 0.76 or greater than 0.78 or greater than 0.80 or greater than 0.82 or greater than 0.84 or greater than 0.86 or greater than 0.87 or greater than 0.88 or greater than 0.89 or greater than 0.90 or greater than 0.91 or greater than 0.92 or greater than 0.93 or greater than 0.94 or greater than 0.95 or greater than 0.96 or greater than 0.97 or greater than 0.98 or greater than 0.99 or greater than 1.00 or greater than 1.01 or greater than 1.03 or greater than 1.05 or greater than 1.07 for histidine, isoleucine, leucine, lysine, methionine+cysteine, phenylalanine+tyrosine, threonine, tryptophan, and valine, and this list can also be considered to describe the essential amino acids for humans.

The composition can also contain branched-chain amino acids (leucine, isoleucine, and valine) in high amounts. In some embodiments the composition can also contain phenylalanine and tyrosine and/or methionine and cysteine in high amounts.

The protein material can be used as food or food ingredient for humans and/or animals, including domesticated or companion animals such as, for example, horses, cattle, bovines, ruminants, hogs, pigs, swine, sheep, goats, turkeys, chickens, or other fowl, cats, dogs. In various embodiments the food or food ingredient contains all amino acids essential for humans and/or domesticated animals and/or pets.

The protein compositions of the invention have the further advantage of lacking allergens. In various embodiments the compositions lack human allergens such as soy allergens, peanut or nut allergens, egg allergens, wheat allergens, pea allergens, dairy allergens, milk allergens, whey allergens, fish allergens, shellfish allergens, or any subset of them. Thus, the protein composition does not contain any of the human allergens recited herein at or above the lowest observed adverse effect level for said allergens, and the level of any or all of these allergens can be zero. The specific allergen level depends on the particular allergen involved and the person of ordinary skill in the art can readily determine from the scientific literature and medical knowledge what the lowest observed adverse effect level is for any particular allergen. In various embodiments the allergen can be a peanut protein, a soy protein, a whey protein, a milk or dairy protein, an egg protein, a nut protein, a pea protein, a wheat protein, a fish protein, or a shellfish protein. In various embodiments the protein compositions of the invention do not contain proteins or materials from any one or more of peanut, milk, soy, nut, egg, whey, wheat, fish, shellfish, or pea, or from any of them. Certain people can have a biological intolerance to any one or more of peanut, milk, dairy products, soy, nut, egg, whey, wheat, fish, shellfish, or pea. This biological intolerance is caused by materials contained in the named dietary compositions. Such intolerance can cause bloating or other digestive disturbances or irregularities, or other physical symptoms known to medical professionals. The protein compositions of the invention are free of or do not contain these materials at a level where the intolerance occurs.

The protein compositions of the present invention have yet another advantage in that they are from reliable sources and are not disrupted by weather, partial or complete crop failures, spikes in demand, or other unpredictable forces. The protein compositions of the present invention can be produced in culture in whatever quantities are desired.

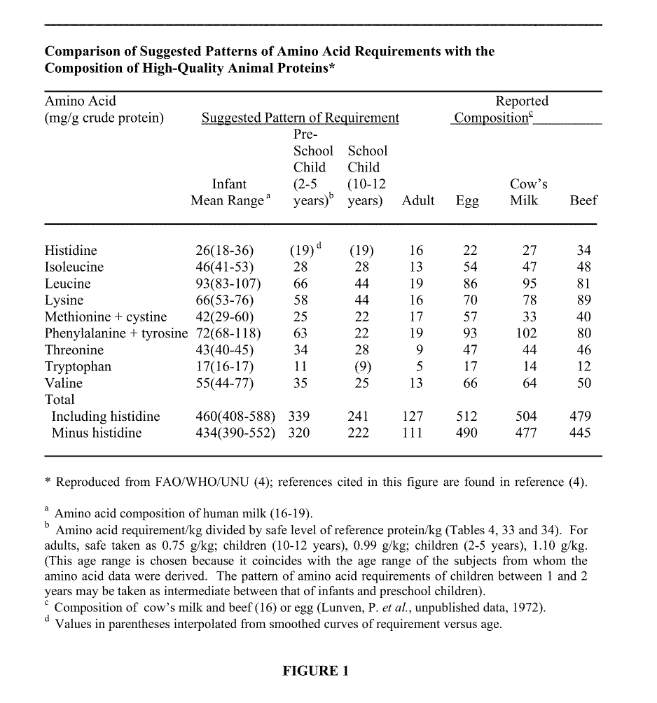

Dietary protein products currently available are limited in one or more of the essential amino acids that cannot be synthesized by human or animal metabolism. For example dairy products are limited in phenylalanine and tyrosine. Legumes are limited in the sulfur-containing amino acid methionine. Grains, such as wheat and corn, are limited in lysine, and can also be limited in threonine (wheat), or tryptophan (corn). Nuts and seeds are often limited in lysine. The Food and Agricultural Organization (FAO) of the United Nations issues recommendations on protein requirements in health and disease for all age groups, as well as recommendations on protein quality. In various embodiments the protein compositions of the invention advantageously contain all essential amino acids in excess of the FAO recommended requirements for 2-5 year old children. This advantage is not found in other plant-derived protein compositions. Thus, in some embodiments the protein compositions of the invention contain an amount of histidine, isoleucine, leucine, lysine, methionine+cysteine, phenylalanine+tyrosine, threonine, tryptophan, and valine, each in an amount that meets or exceeds the FAO recommended requirements for a 2-5 year old child. In various embodiments the protein compositions of the invention provide an amount of any one or more of, or any combination of, histidine, isoleucine, leucine, lysine, methionine+cysteine, phenylalanine+tyrosine, threonine, tryptophan, and valine, in an amount that meets or exceeds the FAO recommended requirements for a 2-5 year old child. In one embodiment the FAO recommended requirements are those listed in FIG. 1 for any one or more of the amino acids or pairs of amino acids listed.

Yet another advantage of the protein compositions of the invention is that they can be properly labeled as vegetarian, vegan, and non-GMO (genetically modified organism) since they qualify under the food descriptions in each of those categories. For example, the compositions can be legally labeled as such under current regulations in the United States, the European Union, China, Japan, and other countries. The protein compositions of the invention are vegetarian because they contain no products or portion of any animal, fish, or fowl or shellfish. The protein compositions of the invention are also vegan because they contain no products or portion of any animal, fish, fowl, dairy products, or eggs. The protein compositions of the present invention are non-GMO because they are produced without the use of recombinant DNA or organisms containing recombinant DNA. The organisms from which the protein compositions are derived from natural sources and contain no recombinant DNA.

Current sources of protein lack one or more of the essential amino acids, or otherwise supply amino acids in quantities that are not nutritionally balanced. One solution to this problem has been to combine proteins from different sources, for example from two or more plant or other sources. In some embodiments the protein composition of the invention is made from a single source, meaning that the protein is derived substantially from one source and not from the combining of proteins from different sources. In one embodiment the single source can be biomass derived from the culturing (e.g. a fermentation) of a single organism or mixture of organisms. By being derived from a source is meant the protein material was purified from, is produced by, or otherwise extracted from the source. By being substantially derived from a source is meant that at least 80% or at least 85% or at least 90% or at least 95% or at least 96% or at least 97% or at least 98% or at least 99% of the protein material was purified from, is produced by, or otherwise extracted from the source. In some embodiments the culturing of any algal and/or microbial biomass is a single source. Protein compositions from a single source do not include combinations of proteins derived from distinct sources, such as distinct plants, animals, or their byproducts that supply different quantities or a different balance of amino acids in the protein produced to the extent that the additional proteins materially change the amino acid or nutritional profile of the protein composition. The protein can also be a protein that is not derived from or contain a fusion protein produced as a result of genetic engineering. For example, adding to protein derived from cellular biomass a protein, peptide, or amino acid material derived from soy, peanut, milk, egg, whey, nut, wheat, fish, shellfish, pea, or other distinct protein sources, which would materially change the amino acid and nutritional profile of the composition, does not produce a protein composition from a single source. Also, a protein composition derived from two or more of soy, peanut, milk, egg, whey, nut, wheat, fish, shellfish, pea, or other distinct protein sources is not from a single source.

Additional advantages of the compositions of the invention are that they do not contain undesirable components that limit their functionality. For example, in some embodiments the compositions of the invention do not contain chlorophyll, which can be found in Spirulina and Chlorella products, and which limits their use in processed foods because of an undesirable appearance in color and poor consumer acceptance. In another embodiment the protein composition does not contain chlorophyll in an amount detectable by the unaided eye and that would materially change the color of the protein composition.

### Proximate Analysis

Proximate analysis is a measure of a food ingredient's nutritional value and involves the partitioning of the food ingredient into six categories based on the chemical properties of the compounds. It generally duplicates animal digestion and describes the energy and nutritional content of the food ingredient. The six categories are: 1. Moisture, 2. Ash, 3. Crude Protein (or Kjeldahl protein), 4. Crude lipid, 5. Crude fibre, and 6. Digestible carbohydrates (or nitrogen-free extracts).

Any of the proteinaceous food or food ingredients can have a total protein content of at least 50% or at least 60% or at least 65% or at least 68% or at least 70% or at least 72% or at least 75% or at least 78% or at least 80% or at least 85% or at least 90%, or from 50% to 70%, or from 65-75%, or from 70-80%, or from 70-85% or from 75-80% or from 75-85%, or from 70-90%, or from 65-90%, or from 75-90%, or from 75-100%, or from 90-100%, all w/w.

In any of the compositions the ash content can be less than about 12% or less than 11% or less than 10% or less than about 9% or less than about 8% w/w or less than about 7% w/w or less than about 6% w/w or from about 3% to about 7% (w/w), or from about 4% to about 6% (w/w), or from about 5% to about 7% (w/w).

Any of the proteinaceous food or food ingredients (or protein composition) of the invention can have varied lipid content such as, for example, about 5% lipid or about 6% lipid or about 7% lipid, or about 8% lipid or less than 30% lipid content or less than 25% lipid content or less than 20% lipid or less than 18% lipid or less than 15% lipid or less than 12% lipid or less than 10% lipid or less than 9% lipid or less than 8% or less than 7% or less than 6% or less than 5% lipid or less than 4% lipid or less than 3% lipid or less than 2% lipid or less than 1.5% lipid or less than 1% lipid or less than 0.75% lipid or less than 0.6% lipid or less than 0.5% lipid, or from about 1% to about 5% lipid, or from about 1% to about 3% lipid, or from 2% to about 4% lipid, all w/w. Lipid content can be conveniently expressed as a fatty acid methyl ester (FAME) profile.

Similarly, any of the proteinaceous food or food ingredients or protein compositions of the invention can have less than 2% or less than 1.0% or less than 0.75% or less than 0.60% or less than 0.50% oil content. The proteinaceous food or food ingredients of the invention thus offer a significant advantage since they can have a UCLAA score above 0.88 or above 0.94 or as otherwise described herein, and have a total protein content of at least 73% or at least 75% or at least 78%, and yet still have a lipid and/or oil content of less than 5% or less than 4% or less than 3% or less than 2% or less than 1.5% or less than 1% or less than 0.05%, or as otherwise described herein.

In some embodiments the protein composition of the invention is not a whole cell composition, i.e., does not contain whole cells. Instead, utilizing the processing techniques described herein a protein product can be obtained having the recited components but not contain whole cells, although in some embodiments depending on how rigorously the processing is applied the composition could contain less than 10% whole cells or less than 7% whole cells or less than 5% whole cells, or less than 4% or less than 3% or less than 2% or less than 1% whole cells, w/w. Additionally, as described herein, the composition can be organoleptically acceptable and have the protein and/or lipid contents stated herein.

In different embodiments non-protein nitrogen content can be less than 12% or less than 10% or less than 8% or less than 7% or less than 6% or less than 5% or less than 4% or less than 3% or less than 2% or less than 1% or less than 0.75% or less than 0.60% or less than 0.5% or from about 1% to about 7% or from 2% to about 6% (all w/w) in any of the proteinaceous food or food ingredients. The non-protein nitrogen can be inorganic nitrogen. The protein compositions of the invention can also have less than 5% or less than 4% or less than 3% or less than 2% or less than 1% or less than 0.75% or less than 0.60% or less than 0.5% or less than 0.25% or less than 0.10% of organic nitrogen, or even no organic nitrogen.

In any of the embodiments the protein compositions of the invention can have a moisture content of less than 20% or less than 15% or less than 12% or less than 10% or less than 9% or less than 8% or less than 7% or less than 6% or less than 5% or less than 4% or less than 3% or less than 2% or less than 1% w/w.

Any of the protein compositions of the invention can comprise at least 75% or at least 78% or at least 80% or at least 81% protein component or as described herein, and less than 10% or less than 7% or less than 5% or less than 3% or less than 2% or less than 1% lipid content or as described herein. In a specific embodiment the composition has at least 65% protein and less than 5% lipid. In other specific embodiments the composition has more than 78% or more than 80% protein and less than 2% or less than 1% lipid component (w/w).

In various embodiments the food or food ingredient can contain any of the stated amounts of protein in combination with any of the stated amounts of lipid. The lipid content of the proteinaceous food or food ingredient can be manipulated as explained herein depending on the source of the protein material and the uses of the protein material to be produced, as well as by varying the steps in its production. The lipid content in the food or food ingredient can be provided, either partially or completely by at least 50% or at least 60% or at least 70% or at least 80% or at least 90% w/w polyunsaturated fatty acids. The polyunsaturated fatty acids can be any one or more of gamma-linolenic acid, alpha-linolenic acid, linoleic acid, stearidonic acid, eicosapentaenoic acid, docosahexaenoic acid (DHA), and arachiconic acid, in any combinations.

In various embodiments any of the protein compositions can contain at least 70% or at least 80% or at least 90% polypeptides of a length of 50 amino acid residues or greater, or 100 amino acid residues or greater, or 200 amino acid residues or greater. The protein compositions of the invention can have protein of an average molecular weight of at least 15 kDa or greater or at least 18 kDa or greater or at least 20 kDa or greater or at least 22 kDa or greater or at least 25 kDa or greater or 15-25 kDa or 15-50 kDa or 15-100 kDa or 15-200 kDa. In other embodiments at least 50% or at least 60% or at least 70% or at least 75% or at least 80% of the proteins in the protein compositions of the invention have a molecular weight of at least 15 kDa or greater or at least 18 kDa or greater or at least 20 kDa or greater or at least 22 kDa or greater or at least 25 kDa or greater or 15-25 kDa or 15-50 kDa or 15-100 kDa or 15-200 kDa. Any of the protein compositions of the invention can also have a water holding capacity (WHC) value of less than 11.0 or less than 10.5 or less than 10.0 or less than 9.5 or less than 9.0.

The protein composition of the invention can be utilized in a wide variety of foods. It can be used either as a supplement or a food substitute. As examples, the protein composition can be utilized or incorporated within cereals (e.g. cereals or breakfast cereals containing mostly grain content), snack bars (a bar-shaped snack containing mostly proteins and carbohydrates), nutritional or energy bars (a bar-shaped food intended to supply nutrients and/or boost physical energy, typically containing a combination of fats, carbohydrates, proteins, vitamins, and minerals), canned or dried soups or stews (soup: meat or vegetables or a combination thereof, often cooked in water; stew: similar to soup but with less water and cooked at lower temperature than soup), as a binder for bulk and/or artificial meats (artificial meats are protein rich foods, usually based on soy or plant proteins, but having no real meat of animal origin in them, but they have characteristics associated with meat of animal origin), cheese substitutes, vegetable “burgers”, animal or pet feed (e.g. in animal or livestock feed for consumption by domesticated animals and/or pets—these feeds can be mostly grain products), and much more. It can also be a nutritional supplement such as a protein or vegetable protein powder. The protein material can also be converted into a food ingredient, e.g., a protein rich powder useful as a substitute for grain-based flour. The protein materials are useful as food ingredients or as foods for both human and animal consumers. In addition to providing an advantageous source of protein the proteinaceous material of the invention can also contain other nutrients, which can be added, such as lipids (e.g., omega-3 and/or omega-6 fatty acids), fiber, a variety of micronutrients, B vitamins, iron, and other minerals being only some examples. These nutrients can be provided in recommended daily amounts, or a multiple thereof, per FDA or other government agency guidelines.

### Biomass

The algal or microbial organisms that are useful in producing the biomass from which the protein material of the invention is obtained can be varied and can be any algae or microbe that produces a desired protein-containing product. In some embodiments the organisms can be algae (including those classified as “chytrids”), microalgae, Cyanobacteria, kelp, or seaweed. The organisms can be either photosynthetic or phototrophic or heterotrophic, or a combination thereof. The organisms can be either naturally occurring or can be engineered to increase protein content or to have some other desirable characteristic. In various embodiments the biomass utilized in the invention can be derived from microbial sources or algal sources (e.g. chytrid biomass) or any suitable source. In different embodiments algae and/or cyanobacteria, kelp, and seaweed of many genera and species can be used, with only some examples being those of the genera Arthrospira, Spirulina, Coelastrum (e.g., proboscideum), macro algae such as those of the genus Palmaria (e.g., palmata) (also called Dulse), Porphyra (Sleabhac), Phaeophyceae, Rhodophyceae, Chlorophyceae, Cyanobacteria, Bacillariophyta, and Dinophyceae. The alga can be microalga (phytoplankton, microphytes, planktonic algae) or macroalga. Examples of microalga useful in the invention include, but are not limited to, Achnanthes, Amphiprora, Amphora, Ankistrodesmus, Asteromonas, Boekelovia, Bolidomonas, Borodinella, Botrydium, Botryococcus, Bracteococcus, Chaetoceros, Carteria, Chlamydomonas, Chlorococcum, Chlorogonium, Chlorella (e.g. Chlorella pyrenoidosa, C. kessleri, C. vulgaris, C. protothecoides), Chroomonas, Chrysosphaera, Cricosphaera, Crypthecodinium sp., Cryptomonas, Cyclotella, Dunaliella, Ellipsoidon, Emiliania, Eremosphaera, Ernodesmius, Euglena, Eustigmatos, Franceia, Fragilaria, Fragilariopsis, Galdieria sp., Gloeothamnion, Haematococcus (e.g., pluvialis), Halocafeteria, Hantzschia, Heterosigma, Hymenomonas, Isochrysis, Lepocinclis, Micractinium, Monodus, Monoraphidium, Nannochloris, Nannochloropsis, Navicula, Neochloris, Nephrochloris, Nephroselmis, Nitzschia, Ochromonas, Oedogonium, Oocystis, Ostreococcus, Parachlorella, Parietochloris, Pascheria, Pavlova, Pelagomonas, Phceodactylum, Phagus, Picochlorum, Platymonas, Pleurochrysis, Pleurococcus, Porphyridium, Prototheca, Pseudochlorella, Pseudoneochloris, Pseudostaurastrum, Pyramimonas, Pyrobotrys, Scenedesmus (e.g., obliquus), Schizochlamydella, Skeletonema, Spyrogyra, Stichococcus, Tetrachlorella, Tetraselmis, Thalassiosira, Tribonema, Vaucheria, Viridiella, Vischeria, and Volvox.

In some embodiments the cells or organisms comprising the biomass of the invention can be any microorganism of the class Labyrinthulomycetes. While the classification of the Thraustochytrids and Labyrinthulids has evolved over the years, for the purposes of the present application, “labyrinthulomycetes” is a comprehensive term that includes microorganisms of the orders Thraustochytrid and Labyrinthulid, and includes (without limitation) the genera Althornia, Aplanochytrium, Aurantiochytrium, Botryochytrium, Corallochytrium, Diplophryids, Diplophrys, Elina, Japonochytrium, Labyrinthula, Labryinthuloides, Oblongochytrium, Pyrrhosorus, Schizochytrium, Thraustochytrium, and Ulkenia. In some examples the microorganism is from a genus including, but not limited to, Thraustochytrium, Labyrinthuloides, Japonochytrium, and Schizochytrium. Alternatively, a host labyrinthulomycetes microorganism can be from a genus including, but not limited to Aurantiochytrium, Oblongichytrium, and Ulkenia. Examples of suitable microbial species within the genera include, but are not limited to: any Schizochytrium species, including Schizochytrium aggregatum, Schizochytrium limacinum, Schizochytrium minutum; any Thraustochytrium species (including former Ulkenia species such as U. visurgensis, U. amoeboida, U. sarkariana, U, profunda, U. radiata, U. minuta and Ulkenia sp. BP-5601), and including Thraustochytrium striatum, Thraustochytrium aureum, Thraustochytrium roseum; and any Japonochytrium species. Strains of Thraustochytriales particularly suitable for the presently disclosed invention include, but are not limited to: Schizochytrium sp. (S31) (ATCC 20888); Schizochytrium sp. (S8) (ATCC 20889); Schizochytrium sp. (LC-RM) (ATCC 18915); Schizochytrium sp. (SR21); Schizochytrium aggregatum (ATCC 28209); Schizochytrium limacinum (IFO 32693); Thraustochytrium sp. 23B ATCC 20891; Thraustochytrium striatum ATCC 24473; Thraustochytrium aureum ATCC 34304); Thraustochytrium roseum (ATCC 28210; and Japonochytrium sp. L1 ATCC 28207. For the purposes of this invention all of the organisms mentioned herein, including the chytrids, are considered “algae” and produce “algal biomass” when fermented or cultured. But any cells or organisms that produce a microbial biomass that includes a desired protein can be utilized in the invention.

In still further embodiments the microbial organism can be oleaginous yeast including, but not limited to, Candida, Cryptococcus, Lipomyces, Mortierella, Rhodosporidium, Rhodotortula, Trichosporon, or Yarrowia. But many other types of algae, cyanobacteria, kelp, seaweed, or yeast can also be utilized to produce a protein rich biomass. These are not the only sources of biomass since biomass from any source can be used that contains desired proteinaceous material of significant nutritional value.

When phototrophic algae are used as the biomass it is advantageous to apply additional steps to produce the protein concentrate. Cellulytic enzymes can be used to assist in deconstructing the cell wall to liberate lipids, carbohydrates, and proteins from each other for enhanced separation and a final product devoid of lipids and carbohydrates. Different solvents, salinities, and pH conditions can be used to remove chlorophyll and other pigments.

In some embodiments the protein compositions of the invention are sourced from biomass, for example algal or microbial biomass, either of which can be phototrophic or heterotrophic. Biomass material is that biological material derived from (or having as its source) living or recently living organisms. Algal biomass is derived from algae and microbial biomass is derived from microorganisms (e.g. bacteria, unicellular yeast, multicellular fungi, or protozoa). The term “cellular biomass” indicates algal and/or microbial biomass. The algae or microbes that produce the protein composition in the biomass can be fermented or amplified in any suitable manner. Biomass utilized in the present invention can be derived from any organism or class of organisms, and examples are described herein such as, for example, heterotrophic algae (e.g. chytrids), or phototrophic or photosynthetic algae. Cellular biomass can be harvested from natural waters or cultivated. Biomass can also be derived from kelp or seaweed. The organisms can be either single cellular or multi-cellular organisms. When cultivated, this can be done in open ponds or in a photobioreactor or fermentation vessels of any appropriate size. The microbes or algae can be either photosynthetic or heterotrophic. Heterotrophic organisms are those that cannot fix carbon and require organic carbon for growth. In some embodiments the biomass is derived from chemotrophic algae, which does not use light for energy but uses chemical energy (a chemoheterotroph). In some embodiments only light and carbon dioxide are provided but nutrients can be included in any culture medium, for example nitrogen, phosphorus, potassium, and other nutrients. In other embodiments sugars (e.g. dextrose) and other nutrients such as salts (e.g., Na₂SO₄, CaCl₂, (NH₄)₂SO₄), and other nutrients (e.g., trace metals) are included in the culture medium depending on the specific needs of the culture.

When sufficient biomass, has been generated the biomass can be harvested from cultivation. The harvest can be taken or made into the form of a broth, suspension, or slurry. The biomass can generally be easily reduced by centrifugation to a raw biomass of convenient volume.

### Organoleptic Properties

Any of the proteinaceous food or food ingredients or protein compositions of the invention can have organoleptic taste and smell properties that are acceptable to humans or to animals. Acceptable properties can be evaluated in comparison to a standard protein, such as whey or pea or soy, or another suitable standard protein. A protein composition having taste and smell properties approaching (or almost as good), comparable to, equal to, or better than the standard as evaluated in organoleptic evaluations is considered to have acceptable properties. A protein composition is comparable to the standard if it is close or similar in its organoleptic properties. A composition having acceptable organoleptic properties also indicates the composition is suitable for use as a food or food ingredient, not merely to a niche consumer that consumes the composition for a special purpose and is willing to tolerate some unpleasant organoleptic properties to achieve their purpose, but for more broad and general nutritional purposes. For example, some algal compositions are consumed by niche consumers for special purposes but these compositions have poor organoleptic taste and smell properties and are not broadly appealing to consumers as common food or food ingredients. Such compositions are therefore not organoleptically acceptable.

Organoleptic taste and smell properties refers to those properties of a food or food ingredient relating to the sense of taste and/or smell, respectively, particularly with reference to the taste and/or smell property being pleasing or unpleasant to a human or animal consumer. Methods of evaluating and quantifying the organoleptic taste and/or smell properties of foods are known by those of ordinary skill in the art. This evaluation enables one to place a particular food or food ingredient on an organoleptic scale indicating a more or less desirable taste and/or smell property relative to another food or food ingredient.

Generally, these methods involve the use of a panel of several persons, for example an evaluation panel of 3 or 4 or 5 or 3-5 or 6 or 7 or 8 or 9 or at least 3 or at least 4 or at least 5 or at least 6 or at least 7 or at least 8 or more than 9 persons. As further examples panels can also include 11 or 15 or 19 persons. The panel is generally presented with several samples to be evaluated (e.g., 3 or 4 or 5 or 6 or 7 or 8 or more than 8 samples) in a “blind” study where the panel members do not know the identity of each sample. The samples can be proteinaceous material derived from cellular biomass. The panel then rates the samples according to a provided scale, which can have 3 or 4 or 5 or 6 or more than 6 categories describing the taste and/or smell properties of each sample. The findings of panel members (e.g. a majority) can then be utilized to determine whether a food sample has more or less desirable organoleptic properties relative to other food samples provided (e.g. a protein standard). The categories can be correlated to more or less desirable organoleptic properties and can be comprised on an organoleptic scale. A sample scoring in one category is considered to have more or less desirable organoleptic properties than a sample scoring in another category. In some embodiments the proteinaceous material in the unprocessed biomass has unacceptable or undesirable organoleptic taste and smell properties, but the properties can be improved by applying the methods described herein. The proteinaceous component can include the protein portion and any lipidic or other component that is covalently or otherwise closely associated with the protein component as described herein.

In some studies a “standard” food or proteinaceous material as known in the art can be included to represent an acceptable organoleptic profile—i.e. taste and smell properties. Those samples rating similar to, equivalent to, or higher than the standard are organoleptically acceptable or desirable while those rating lower are unacceptable or undesirable. In various embodiments the standard can be soy or whey or pea protein, or any suitable standard under the specific circumstances. It is well known in the art how to prepare these standards for evaluation in organoleptic tests.

One example of such a method of evaluating such properties of food is the 9 point hedonic scale, which is also known as the “degree of liking” scale. (Peryam and Girardot, N. F., Food Engineering, 24, 58-61, 194 (1952); Jones et al. Food Research, 20, 512-520 (1955)). This method evaluates preferences based on a continuum and categorizations are made based on likes and dislikes of participating subjects. The 9 point method is known to persons of skill in the art, and has been widely used and shown to be useful in the evaluation of food products. The 9 point hedonic scale includes categories of 1. Like extremely, 2. Like very much, 3. Like moderately, 4. Like slightly, 5. Neither like nor dislike, 6. Dislike slightly, 7. Dislike moderately, 8, Dislike very much, and 9. Dislike extremely. One can therefore evaluate whether certain foods have more desirable or less desirable taste and/or smell properties. Acceptable taste and smell properties can also be evaluated according to the hedonic scale. In one embodiment the protein food or food ingredient produced by the methods of the present invention scores higher on the 9 point hedonic scale versus protein products from the same source that has not been subjected to one or more steps of the invention. In other embodiments the proteinaceous food or food ingredients or protein compositions of the invention score at least 4 or at least 3 or at least 2 on the 9 point hedonic scale when evaluated by a panel as described herein. Other methods of evaluating organoleptic taste and/or smell properties can also be utilized.

The specific criteria utilized by an evaluation panel can vary but in one embodiment the criteria include whether the organoleptic properties of a sample are generally pleasing or displeasing. Thus, in one embodiment a sample can be rated as having generally pleasing organoleptic properties at least equivalent to a standard. Other common criteria that can be evaluated include, but are not limited to, whether the sample has a smell or taste that is briny (having a salty or salt water character), fishy (having a character related to fish), or ammonia-like (having a character related to or resembling ammonia). Any one or more of these properties can be evaluated. These can be subjective determinations but people are familiar with these sensations and, when provided to a panel of persons to evaluate, meaningful conclusions are generated. Other criteria that can be used are the general organoleptic taste and smell properties of the sample indicated by whether the sample has more pleasing, less pleasing, or is about the same as a standard sample provided. Utilizing known methods of evaluating proteins statistically meaningful conclusions can be readily reached, as is commonly done in the art.

The organoleptic properties of a protein material relate directly to the physical composition of the material. Certain chemicals that cause undesirable organoleptic properties are removed by the methods described herein, which result in a markedly different protein composition than that originally present in the biomass. These chemicals can be one or more of a number of malodorous and/or foul tasting compounds, which in some cases are volatile compounds. Without wanting to be bound by any particular theory examples of compounds believed to contribute to undesirable organoleptic properties include lipidic compounds, including saturated or unsaturated or polyunsaturated fatty acids (e.g., DHA) and their breakdown products, lysophospholipids, aldehydes (e.g. those produced by oxidation of lipids), and other breakdown products. These fatty acids or their breakdown products can also become oxidized (perhaps during isolation and/or purification of a proteinaceous material) and such compounds give unpleasant organoleptic properties to a food or food ingredient.

In some embodiments the compounds that confer undesirable organoleptic properties are lipidic material, which can be covalently bound to desired proteins or otherwise closely associated with the protein content of the material. Lipidic compounds can also be non-covalently bound but nevertheless closely associated with the protein in such a way that they cannot be purified way from the protein by conventional purification methods. The chemicals can also be saturated or unsaturated fatty acid moieties. The fatty acid (or fatty acid moieties) can comprise but are not limited to gamma-linolenic acid, alpha-linolenic acid, linoleic acid, stearidonic acid, eicosapentaenoic acid, docosahexaenoic acid (DHA), and arachiconic acid, any ω-3 or ω-6 fatty acid, a breakdown product of any of them, or any of the aforementioned in an oxidized form. The methods of the invention can reduce the amount of one or more of these compounds in the protein material by at least 20% or at least 30% or at least 40% or at least 50% or at least 70% or at least 80% or at least 90% or at least 95% or at least 97% or at least 99% versus the amount in protein material from the biomass that has not been subjected to a method of the invention. Malodorous and/or foul tasting compounds (organoleptically unacceptable compounds) can also include oxidized lipids (e.g., oxidized unsaturated fatty acids or oxidized omega-3 fatty acids, for example any of those described above) as well as proteins that can confer the malodorous and/or foul tasting properties. Malodorous and/or foul tasting compounds can also comprise lipidic material covalently bound to or otherwise closely associated with proteins in the proteinaceous material. Chemicals causing undesirable organoleptic properties can also be enzymatic or chemical breakdown products of lipid molecules, for example any of the lipid molecules described herein. In some embodiments the microbial biomass contains a protein or proteins having unacceptable or undesirable organoleptic properties. When processed according to the invention the protein (or proteins) having unacceptable or undesirable organoleptic properties can be removed, converted, or changed into a protein (or proteins) having acceptable or desirable organoleptic properties.

### Defined Medium

In some embodiments the protein material of the invention is produced by incubating or fermenting biomass in a defined medium to produce a cellular biomass. Rich growth media typically have copious amounts of organic nitrogen, such as yeast extract and peptone. Defined media are obtained by reducing or eliminating components containing organic nitrogen. In various embodiments the defined media contain dextrose and salts, such as ammonium sulfate, sodium chloride, and trace metals. The person of ordinary skill in the art will readily realize that the specific composition of a defined medium can be varied depending on the application. By performing growth in a defined medium and by performing the methods described herein a more nutritionally balanced protein product can be obtained from microbial or algal biomass. Defined media can contain inorganic nitrogen, for example nitrogen salts. Various defined media can be made using one or more of the following components provided as described below:

TABLE 1

| Component | Amount | |
|---|---|---|
| NaCl | 1.0-10.0 | g/L |
| CaCl | 0.05-1.0 | g/L |
| Na₂SO₄ | 1-10.0 | g/L |
| (NH₄)-salt | 0.1-6.0 | g/L |
| KCl | 0.05-5.0 | g/L |
| MgSO₄7H₂O | 0.5-10.0 | g/L |
| Antifoam (KFO) | 0-10 | ml/L |
| Glucose | 1.0-100 | g/L |
| KPO4 monobasic | 0.5-10.0 | g/L |
| EDTA | 1.0-10,000 | mg/L |
| Boric acid | 1.0-500 | mg/L |
| Trace minerals soln | 2.0-20.0 | ml/L |
| Biotin | 0.1-100 | ug/L |
| Thiamine | 1.0-10,000 | ug/L |
| Vitamin B12 | 1.0-1000 | ug/L |
| NO₃-salt | 0.1-6.0 | g/L |

In various embodiments the defined medium can contain less than 20% w/w organic nitrogen or less than 15% w/w organic nitrogen or less than 10% or less than 7% or less than 5% or less than 2% or less than 1% or less than 0.5% or less than 0.25% or less than 0.01% w/w organic nitrogen. In one embodiment the defined medium does not contain organic nitrogen. It was discovered unexpectedly that by cultivating the organisms described herein in a defined medium as described herein the protein composition produced by the methods has a UCLAA score for essential amino acids of 0.85 or greater or 0.88 or greater or 0.90 or greater or 0.92 or greater or 0.95 or greater or 0.96 or greater or 0.97 or greater or 0.98 or greater or 0.99 or greater or greater than 1.0 or greater than 1.01 or greater than 1.02 or greater than 1.03 or greater than 1.04 or greater than 1.05 or greater than 1.06 or greater than 1.07, as described herein. The use of the defined medium therefore resulted in cellular biomass producing a protein composition having an amino acid profile that provides higher quality nutrition for humans and animals. Organic nitrogen can come from, but is not limited to, one or more of following sources: yeast extract, brain heart infusion broth, casein hydrolysate, lactalbumin hydrolysate, soybean hydrolysate, gelatin hydrolysate, beef heart hydrolysate, sodium glutamate, peptone, tryptone, or phytone.

In various embodiments the defined medium can also contain inorganic nitrogen in amounts of less than 8 g/L or less than 6 g/L or less than 5 g/L or less than 4 g/L or less than 3 g/L or less than 2 g/L or less than 1 g/L or less than 0.75 g/L or less than 0.50 g/L. In other embodiments the defined medium can contain 0.25-10.0 g/L of inorganic nitrogen or 0.25-8.0 g/L or 0.25-5.0 g/L or 1.0-10.0 g/L or 1-8 g/L or 1-5 g/L of inorganic nitrogen. In various embodiments the inorganic nitrogen can be provided in the form of ammonium salts, urea, or salts of nitrates or nitrites.

Many versions of the defined medium can function in the invention, and herein are listed only some examples. In certain embodiments the defined medium can be made using the following components: between 3.0-9.0 g/L of NaCl, 0.25-0.9 g/L of CaCl, 2.0-8.0 g/L of Na₂SO₄, 2.0-8.0 g/L of NH₄ salt and/or 0.1-4.0 g/L of NO₃ salt, 0.25-2.0 g/L of KCl, 1.5-8.0 g/L of MgSO₄7H₂O, 0.5-8 ml/L of Antifoam (KFO), 5-75 g/L of Glucose, 1.0-8.0 g/L of KPO₄ monobasic, 10-80 mg/L of EDTA, 20-350 mg/L of Boric acid, 3.0-18.0 ml/L of trace minerals solution, 1-75 ug/L of Biotin, of 5-2500 ug/L of Thiamine, and 0.5-500 ug/L of Vitamin B12. In such defined medium the NH₄ salt can be selected from, for example, (NH₄)₂SO₄, NH₄Cl, (NH₄)₂CO₃, NH4NO₃ or any other ammonium salt. In such defined medium the NO₃ salt can be for example, NaNO₃, KNO₃, NH₄NO₃, or any other NO₂ or NO₃ salt.

In certain embodiments the defined medium can be made using the following components: between 4.0-8.0 g/L of NaCl, 0.3-0.9 g/L of CaCl, 3.0-7.0 g/L of Na₂SO₄, 3.0-7.0 g/L of NH₄-salt and/or 0.25-2 g/L of NO₃-salt, 0.25-1.0 g/L of KCl, 2.0-6 g/L of MgSO₄7H₂O, 0.5-5.0 ml/L of Antifoam (KFO), 5.0-50 g/L of Glucose, 1.0-7.0 g/L of KPO₄ monobasic, 25-75 mg/L of EDTA, 25-200 mg/L of Boric acid, 4.0-15 ml/L of trace minerals solution, 0.5-50 ug/L of Biotin, of 50-1000 ug/L of Thiamine, and 0.5-50 ug/L of Vitamin B12. In such defined medium the NH₄-salt can be selected from, for example, (NH₄)₂SO₄, NH₄Cl, (NH₄)₂CO₃, NH₄NO₃ or any other ammonium salt. In such defined medium the NO₃-salt can be for example, NaNO₃, KNO₃, NH₄NO₃, or any other ammonia, nitrate, or nitrite salt.

In other embodiments the defined medium can be made using the following components: between 5.0-8.0 g/L of NaCl, 0.3-0.9 g/L of CaCl, 3.0-6.0 g/L of Na₂SO₄, 0.25-1.5 g/L of NH₄-salt and/or 0.25-2 g/L of NO₃-salt, 0.25-0.55 g/L of KCl, 2.5-4.5 g/L of MgSO₄7H₂O, 0.5-1.5 ml/L of Antifoam (KFO), 10-50 g/L of Glucose, 1.0-4.5 g/L of KPO₄ monobasic, 30-70 mg/L of EDTA, 30-70 mg/L of Boric acid, 5.0-10.0 ml/L of trace minerals solution, 0.5-10 ug/L of Biotin, of 50-250 ug/L of Thiamine, and 0.5-5 ug/L of Vitamin B12. In such defined medium the NH₄-salt can be selected from, for example, (NH₄)₂SO₄, NH₄CL, (NH4)₂CO₃, NH₄NO₃ or any other ammonium salt. In such defined medium the NO₃-salt can be for example, NaNO₃, KNO₃, NH₄NO₃, or any other NO₂ or NO₃ salt.

Among other nutritional benefits, the protein composition obtained by growth of biomass in a defined medium contains a higher proportion of essential amino acids versus the same biomass grown on a rich medium. Protein compositions obtained from a rich medium typically have less than 35% essential amino acids as a percent of total protein. But in various embodiments the protein composition obtained from biomass grown on a defined medium contains greater than 35% essential amino acids or greater than 37% essential amino acids or greater than 40% essential amino acids, or greater than 42% essential amino acids or greater than 44% essential amino acids or greater than 45% essential amino acids or greater than 46% essential amino acids or greater than 47% essential amino acids or greater than 48% or greater than 49% or greater than 50% essential amino acids, all as a percentage of total protein, w/w. In other embodiments the amount of essential amino acids in the protein composition as a percent of total protein is increased by at least 3% or at least 4% or at least 5% or at least 6% or at least 7% or at least 8% or at least 10% or at least 12% or at least 13% or at least 15% or at least 17% or at least 19% or at least 20% when the protein is obtained from biomass grown on a defined medium versus a rich medium.

In some embodiments the protein product obtained by growth in a defined medium contains less than 20% glutamic acid or less than 19% glutamic acid or less than 18% glutamic acid or less than 17% glutamic acid or less than 16% glutamic acid or less than 15% glutamic acid or less than 14% glutamic acid (all as a percentage of total protein), i.e. lower amounts of glutamic acid than when growth is done in a rich medium. Higher amounts of leucine (e.g. more than 4% or more than 4.5% or more than 5.0%) and lower amounts of arginine (e.g. less than 17% or less than 15% w/w) can also be obtained, alone or in combination with the lower amounts of glutamic acid. Growth in a defined medium can also produce a protein product containing more than 4% isoleucine, and/or or more than 7% leucine and/or less than 9% arginine or less than 8% arginine.

Also provided are methods of isolating or deriving the protein material from biomass sources. Any of the protein materials described herein can have phe+tyr in an amount of any of at least 65 mg/g or at least 68 mg/g or at least 70 mg/gm and/or can also have met+cys in an, amount of any of at least 28 mg/g or at least 30 mg/g or at least 32 mg/g or at least 33 mg/g. In some embodiments the protein material of the invention can comprise at least 5% or at least 7% or at least 8% or at least 10% or at least 12% or at least 14% or at least 15% or at least 18% or at least 20% or at least 22% or at least 24% or at least 25% or at least 27% or at least 29% greater amount of phe+tyr and/or met+cys when cultivated in a defined medium as described herein versus a rich medium. In other embodiments the protein compositions of the invention can have at least 3.5% or at least 3.7% or at least 3.9% or at least 4.1% phenylalanine and/or at least 2.9% or at least 3.0% or at least 3.1% or at least 3.2% or at least 3.3% tyrosine. The protein compositions of the invention can also have at least 2.2% or at least 2.3% or at least 2.4% or at least 2.5% methionine and/or at least 0.9% or at least 1.0% or at least 1.1% cysteine or cystine. In one embodiment the protein composition of the invention meets all FAO requirements for UCLAA of essential amino acids for a 2-5 y/o child.

When biomass is processed according to the methods described herein and using a defined medium instead of a rich medium, the protein composition that is yielded has some surprising beneficial properties. The protein composition can have a reduced amount of glutamic acid and arginine, and the percentage of all other amino acids (w/w) is increased versus the rich medium. In various embodiments the percent of glutamic acid is less than 22% or less than 20% or less than 18% or less than 15% or less than 14%. In some embodiments the percentage of arginine is less than 9% or less than 8% or less than 7%.

Another surprising benefit from the cultivation of biomass on a defined medium versus a rich medium is that the portion of branched chain amino acids increases. The branched chain amino acids include leucine, isoleucine, and valine. When cultivated on a defined medium the portion of branched chain amino acids as a percent of total protein can be at least 13% or at least 14% or at least 14.5% or at least 15% or at least 15.5% or at least 16% or at least 17% or at least 18% or at least 19% or at least 20% or at least 21% or at least 22% or at least 23% or at least 24% or at least 25% at least 26% or at least 27% or at least 28% or at least 30% as a percentage of total protein. The portion of leucine can be at least 5.5% or at least 6.0% or at least 6.5% or at least 6.7% as a percentage of total protein. The portion of isoleucine can be at least 3.0% or at least 3.2% or at least 3.4% or at least 3.6% or at least 3.8% as a percentage of total protein. The portion of valine can be at least 4.4% or at least 4.5% or at least 4.6% or at least 4.7% as a percentage of total protein.

In a particular embodiment the protein product obtained by growth in a defined medium contains amino acids as a percent of total protein as follows, containing any one or more of Asp, 9%±1.0% or±2% or greater than 5% or greater than 7% or greater than 8%; Thr, 4%±0.5% or±1% or greater than 3% or greater than 3.5% or greater than 3.7% or greater than 3.9% or greater than 4.0%; Ser, 4.5%±0.5% or±1% or greater than 3% or greater than 3.5% or greater than 4%; Glu, 24%±1.0% or±2% or less than 35% or less than 30% or less than 28% or less than 27% or 20-28% or less than 20% or less than 17% or less than 15% or less than 13% or greater than 10% or 10-15% or 8-15%; Pro, 3.5%±0.5% or ±1% or greater than 3% or greater than 3.5% or greater than 3.7% or greater than 3.9%; Gly, 4.0%±0.3% or at least 3.8% or at least 4% or at least 4.5% or at least 4.7%; Ala, 5%±1.0% or at least 5% or at least 5.5%; Val, 5.0%±0.5% or±1.0% or greater than 4.5% or greater than 5%; Ile, 3.5%±0.5% or at least 3.0% or at least 3.5% or at least 3.7% or at least 4% or at least 4.5%; Leu, 6.8%±1% or±2% or greater than 5.7% or greater than 5.9% or greater than 6.0% or greater than 6.2% or greater than 6.4% or greater than 6.5% or greater than 6.7% or greater than 7% or greater than 7.5% or greater than 8%; Tyr, 3%±0.5% or greater than 2.7% or greater than 2.8% or greater than 2.9% or greater than 3.0% or 2.7-3.0%; Phe, 4%±0.5% or±1% or greater than 3% or greater than 3.4% or greater than 3.5% or greater than 3.7% or greater than 3.8% or 3.0-3.5%; Lys, 6.25%±1.0% or±2% or greater than 4% or greater than 5% or greater than 5.5% or greater than 6.0% or greater than 6.2% or greater than 6.3%; His 2%±0.1% or greater than 1.6% or greater than 1.7%; Arg, 9%±1% or±2% or greater than 5.5% or greater than 6.0% or less than 20% or less than 15%; Cys, 1.4%±0.2% or 1.6%±0.2% or±0.5% or greater than 0.8% or greater than1.0%; Met 2.0%±0.5% or±1% or greater than 1% or greater than 1.5% or greater than 1.7% or greater than 1.9% or greater than 2.0% or greater than 2.2%; Trp 0.8%±0.25% or 1.2%±0.25% or±0.5% or greater than 0.8% or greater than 0.9% or greater than 1.0% or greater than 1.1%. A protein composition of the invention can have any one or more of these quantities of the listed amino acids, or any subset of them. Every possible subset or sub-combination of amino acids and their quantities is disclosed as if set forth fully herein. These values are in an isolated protein composition that contains the low amounts of lipids recited herein, and not whole cell biomass. Therefore, the listed values have a higher bioavailability than compositions of whole cell biomass.

It was also discovered that it is possible to use a defined medium to obtain a higher percentage of particular essential amino acids that might be desirable in a specific application, as disclosed herein. In particular embodiments the protein composition produced by the methods using a defined medium can contain any one or more of the essential amino acids in any of the amounts described above. A protein composition of the invention can have any one or more of these quantities of the listed essential amino acids as disclosed herein, or any subset of them. Every possible subset or sub-combination of amino acids is disclosed as if set forth fully herein.

In other embodiments the protein composition of the invention derived from biomass fermented in a defined medium can have particular amino acid content comprising any one or more of the following, or any possible subcombination thereof: a leucine content of at least 65 mg/g or at least 66 mg/g or at least 67 mg/g or at least 68 mg/g; an isoleucine content of at least 36 mg/g or at least 37 mg/g or at least 38 mg/g; a lysine content of at least 60 mg/g or at least 61 mg/g or at least 62 mg/g or at least 63 mg/g; a valine content of at least 43 mg/g or at least 44 mg/g or at least 45 mg/g or at least 46 mg/g; a phe and tyr combined content of at least 68 mg/g or at least 69 mg/g or at least 70 mg/g or at least 71 mg/g; and a met and cys combined content of at least 32 mg/g or at least 33 mg/g or at least 34 mg/g or at least 35 mg/g. Each possible subset of the above contents is disclosed as if set forth fully herein.

### Color

Another important organoleptic aspect of a food or food ingredient is color. The color of a food or food ingredient is an important quality relating to its desirability as a food or food ingredient from the perspective of the consumer. The protein compositions of the present invention have a color that is principally white or beige on a food coloring chart. In one embodiment the protein composition is white or beige, as determined by standard color charts for foods (e.g. dry milks), but in other embodiments can be within one or two or three or four shades away from white or beige on a standard color chart, In some embodiments the whey color standards chart #100 can be used. The color can also be a uniform color. But persons of ordinary skill in the art will realize other appropriate color standards that can also be used in the invention to evaluate food color, such as those published by the American Dairy Products Institute. In some embodiments a distinct yellowish or greenish color is not an acceptable color.

### Fermentation and Pasteurization

The selected biomass can be fermented in a fermentation broth and conditions desirable for the type of biomass selected. After fermentation one or more steps of washing the pellet can be performed. A step of mechanical homogenization can also be performed. This can be done, for example, by bead milling or ball milling, but other forms of mechanical homogenization can also be used. Some examples of mechanical homogenization include, but are not limited to, grinding, shearing (e.g., in a blender), use of a rotor-stator, a Dounce homogenizer, use of a French press, vortexer bead beating, or even shock methods such as sonication. More than one method can be used to homogenize the biomass.

Pasteurization is a process that destroys microorganisms through the application of heat. It is used in a wide variety of food preparation processes. Pasteurization can involve heating the biomass mixture to a particular temperature and holding it at the temperature for a minimum period of time. The pasteurization step can be accomplished by raising the temperature of the biomass to at least 50° C. or at least 55° C. or about 60° C. or at least 60° C. or at least 65° C. or about 65° C. or at least 70° C. or about 70° C., or from 50-70° C., or from 55-65° C. The mixture can be held at the temperature for at least 10 minutes or at least 15 minutes or at least 20 minutes or at least 25 minutes or 20-40 minutes, or 25-35 minutes or for at least 30 minutes or for about 30 minutes or for at least 35 minutes or at least 40 minutes or 30-60 minutes or for more than 60 minutes. Persons of ordinary skill in the art with resort to this disclosure will realize that pasteurization can also be accomplished at a higher temperature in a shorter period of time. Any suitable method of pasteurization can be used and examples include vat pasteurization, high temperature short time pasteurization (HTST), higher-heat shorter time (HHST) pasteurization, and in line pasteurization. Temperature and time periods can be selected accordingly.

When a pasteurization step is included it can be performed on the biomass subsequent to fermentation and prior to the acid wash step. The acid wash step can be performed subsequent to the pasteurization step. In one embodiment the steps can include a pasteurization step, a homogenization step (e.g., bead milling), and an acid wash step, which can be performed in the stated order. In one embodiment the pasteurization step is performed prior to the homogenization step and/or prior to the acid wash step. In another embodiment the homogenization step is performed subsequent to the pasteurization step. In one embodiment the acid wash step is performed subsequent to the pasteurization step. The acid wash step can be performed either before or subsequent to the homogenization step and/or the pasteurization step. All of the steps can be performed in the order recited and additional steps can be performed before or after, or in between the recited steps. In one embodiment a solvent extraction (or solvent washing) step can be performed subsequent to the acid washing step.

These methods can yield a protein composition that has acceptable or desirable organoleptic properties, even if the biomass is comprised of organisms that produce a proteinaceous material or other materials that have undesirable organoleptic properties. The methods can convert the proteinaceous material derived from the biomass from one having undesirable organoleptic properties into a protein composition that has more desirable or acceptable organoleptic properties, and one that is suitable or acceptable as a food or food ingredient as measured by performing acceptably in an organoleptic evaluation.

### Methods

The methods of the invention are useful for producing the protein compositions of the invention. Microbial and algal biomass sources have undesirable organoleptic taste and smell properties, sharply limiting their use as foods or food ingredients. The methods described herein allow for the conversion of the protein material derived from biomass sources having undesirable organoleptic properties into a protein composition having organoleptic properties acceptable to humans and animals.

The methods of the invention can comprise any one or more or all of the following steps. The methods can comprise a step of fermentation of cellular biomass, such as an algae or micro-algae or microbe to form a microbial or algal biomass; one or more steps of water or solvent washing the biomass; one or more steps of pasteurization of the biomass; one or more steps of lysing and/or homogenization of the cells of the biomass, which can be done by any suitable method (e.g., mechanical homogenization), and can be done in any of the solvents listed herein; one or more steps of delipidation of the biomass, which can be performed in any suitable solvent as described herein and can be optionally done simultaneously with or during the homogenization step; performing one or more steps of an acid wash on the biomass; one or more steps of delipidation or solvent washing (or solvent extraction) of the acid washed biomass; drying of the biomass; optionally passing of the biomass through a particle size classifier; and retrieval of proteinaceous product material. The methods can involve performing the steps in the order listed or in any order, and one or more of the steps can be eliminated. One or more of the steps can be repeated to optimize the yield or quality of protein material from the biomass such as, for example, repetition of one or more delipidation step.

The one or more steps of water or solvent washing the biomass and/or the one or more steps of pasteurization of the biomass, and/or the one or more steps of lysing of the biomass can be done by conventional methods.

### Delipidation and Solvent Washing or Extraction

In some embodiments the methods involve one or more steps of mechanical homogenization or mixing, which can involve (but is not limited to) bead milling or other high shear mixing (e.g. a ROTOSTAT™ mixer) or emulsifying. This can be done on the biomass before or after the (optional) water or solvent washing and before or after a pasteurization step. A homogenization step can be performed for at least 5 minutes or at least 10 minutes or at least 15 minutes or at least 20 minutes. A homogenization step can involve the creation of an emulsion, a suspension, or a lyosol, and can involve particle size reduction and dispersion to provide smaller particles distributed more evenly within a liquid carrier. Homogenization roduces a more uniform or “homogenized” composition, such as a more consistent particle size and/or viscosity of the mixture. These one or more steps can be followed by or separated by a step of centrifugation and (optionally) re-suspension in a buffer or solvent for an (optional) additional step of homogenization or mixing. Other mechanical stressors include, but are not limited to ultrasonic homogenizers or roto/stator homogenizers, or homogenizers that use high speed rotors or impellers.

The biomass can be subjected to one or more delipidation step(s) prior to or after being subjected to an acid wash. The mechanic stress can be applied with the biomass in contact with an appropriate solvent. Thus, delipidation can involve a lipid extraction or solvent washing step. A solvent washing step involves exposure (or “washing”) of the biomass to solvent for an appropriate period of time, which can be at least 5 minutes or at least 10 minutes or at least 15 minutes or about 15 minutes). The solvent can be any appropriate solvent, and in some embodiments is a polar solvent or a polar, protic solvent. Examples of useful polar, protic solvents include, but are not limited to ethanol, formic acid, n-butanol, isopropanol (IPA), methanol, acetic acid, nitromethane, hexane, acetone, water, and mixtures of any combination of them. For example, in one embodiment the solvent can be a combination of hexane and acetone (e.g., 75% hexane and 25% acetone). In another embodiment the solvent in 90% or 100% ethanol. Any suitable ratio of solvent to biomass can be used such as, for example, 5:1, 6:1, 7:1, 8:1, 9:1, and other ratios. But the skilled person will realize other appropriate solvents or combinations that will find use in the invention. In various embodiments a delipidation step can remove at least 10% or at least 25% or at least 35% or at least 50% or at least 70% or at least 75% or at least 80% or at least 90% or at least 95% or at least 97% or at least 98% of the total lipid in the starting material, all w/w.

The procedure should ensure proper lysing of the cells comprising the biomass to maximize the protein extraction and make lipidic material available for extraction from the biomass. After mechanical homogenization the biomass can be separated by centrifugation and the lipidic materials in the supernatant removed. One or more additional steps of delipidation or solvent washing with the solvent can be performed to maximize delipidation. In some embodiments a second or subsequent cycle(s) of delipidation can utilize a different solvent than used in the first cycle or in a previous cycle to increase the chances of removing more undesirable compounds. In some embodiments a second solvent can also be included to provide for separation, for example including hexane and/or acetone or another hydrophobic solvent can provide for separation and thus extract more undesirable hydrophobic compounds. After homogenization and at least one solvent washing step (solvent washing can be done simultaneously with homogenization by homogenizing in the presence of solvent) the mixture or biomass can be referred to as a delipidated biomass. The biomass can also have been subjected to mechanical homogenization as a separate step before the solvent washing steps.

Without wishing to be bound by any particular theory it is believed that compounds having undesirable organoleptic taste and smell properties may be removed in the one or more delipidation or solvent washing step(s) and/or the one or more acid wash step(s) and/or the one or more steps of solvent washing following the one or more acid washing step(s). Additional substances with undesirable organoleptic properties can be removed by repeating any of the steps one or two or three or more than three times. In some embodiments the order of the steps being performed is also useful for removing undesirable organoleptic properties from a final protein composition. The steps and/or the order in which they are performed can convert a protein composition from one that has undesirable organoleptic properties into a protein composition that is organoleptically pleasing and acceptable as a food or food ingredient. Additional processes described herein can also be performed as one or more steps in the methods of making or synthesizing a protein material. The result of the processes is a material that is high in protein content and derived from biomass.

In various embodiments the protein material prepared according to the invention has a reduced lipid content. In some embodiments the methods of the invention reduce the lipid content of the biomass from more than 10% or more than 8% or more than 7% or more than 6% or more to 5% to a protein composition suitable as a food or food ingredient containing less than 5% lipid content or less than 4% lipid content or less than 3% or less than 2% lipid content or less than 1% lipid content, all w/w.

### Acid Wash

In some embodiments the biomass is subjected to one or more acid wash step(s). The acid wash step can be performed on pasteurized and/or delipidated biomass. Acid washing can comprise exposing the delipidated biomass to acid or a depressed pH for a period of time. The biomass, and therefore the proto-protein it contains, can be exposed to the acid wash in a solution, suspension, slurry, or any suitable state. The acid wash can utilize any suitable inorganic acid (or a suitable organic acid), which are derived from one or more inorganic compounds that form hydrogen ions when dissolved in water. Examples include, but are not limited to, sulfuric acid, nitric acid, phosphoric acid, boric acid, hydrochloric acid, hydrofluoric acid, hydrobromic acid, and perchloric acid. The person of ordinary skill will realize other inorganic acids that also function in the invention. The delipidated biomass can be mixed with water to generate an aqueous mixture. The acid solution (e.g., 1M sulfuric acid) can then be pipetted into the mixture until the pH is reduced to a depressed pH. In various embodiments the pH can be adjusted to a depressed pH of about 4.0 or about 3.8 or about 3.5 or about 3.3 or about 3.2 or about 3.0 or about 2.8 or about 2.5 or from about 2.0 to about 2.5 or from about 2.0 to about 3.0, or from about 2.0 to about 4.0, or from about 2.0 to about 3.5, or from about 2.2 to about 2.8, or from about 2.3 to about 2.7, or from about 2.2 to about 3.8, or from about 2.3 to about 3.7, or from about 2.5 to about 3.0, or from about 2.8 to about 3.2, or from about 3.0 to about 3.5, or from about 3.2 to about 3.8. In other embodiments the pH can be adjusted to less than about pH 4.5 or less than about pH 4.0 or less than about pH 3.7 or less than about pH 3.6 or less than about pH 3.5 or less than about pH 3.3 or less than about pH 3.0 or less than about pH 2.7 or less than about pH 2.5. The mixture can then be held at the indicated pH for a period of time. The mixture can also be mixed or stirred or incubated for the period of time, or a portion thereof. The period of time can be any of at least 1 minute or at least 5 minutes or at least 10 minutes or at least 20 min. or at least 30 min, or from about 20 minutes or about 30 minutes, or about 40 minutes, or from 1-15 minutes or from 1-60 minutes or from 10-30 minutes, or from 10-40 minutes, or from 10-60 minutes or from 20-40 minutes, or from 20 minutes to 1 hour, or from 10 minutes to 90 minutes, or from 15 minutes to 45 minutes, or at least 1 hour or about 1 hour or at least 90 minutes or at least 2 hours.

After the biomass has been exposed to the depressed pH for an appropriate period of time (and optional mixing) the pH can then be raised to a raised pH by addition of a basic or alkaline compound, for example KOH. Persons of ordinary skill in the art will realize that other basic or alkaline compounds can also be used, for example sodium hydroxide, calcium hydroxide, or other basic compounds. The basic compound can be added at any convenient concentration, e.g., about 1 M or 0.5-1.5 M or 0.75-1.25M. The basic compound can be added until the pH is adjusted to a raised pH of about 4.5. But in other embodiments the raised pH can be about 4.0 or about 4.2 or about 4.7 or about 5.0. In more embodiments the pH can be raised to greater than 4.0 or greater than 4.2 or greater than 4.5 or greater than 4.7 or greater than 5.0. After the pH adjustment to the raised pH the mixture can be stirred or incubated for an appropriate period of time, which in some embodiments is about 10 minutes or about 15 minutes or about 20 minutes or about 30 min or about 1 hour or about 90 minutes or more than 30 minutes or more than 1 hour or from 10-60 minutes or from 20-60 minutes.

When the pH is adjusted to the depressed pH there is a noticeable decrease in the viscosity of the mixture from a thick slurry of poor mixing capability to a thin, watery consistency of markedly lower viscosity (i.e. there is an observable decrease in viscosity). The decrease in viscosity can be observed at the start of the acid addition by, for example, the inability of a common laboratory overhead mixer to be able to fully blend the solution (cavitation at the impeller). As the pH is lowered the change in viscosity can be observed as changing to a viscosity similar to a watery solution requiring a reduction in the impeller tipspeed to avoid splashing of the solution. Thus, the change in viscosity can be a decrease of at least 10% or at least 20% or at least 30% or at least 40% or at least 50%, as measured by standard methods of measuring viscosity such as a viscometer. Examples of methods of measuring viscosity include, but are not limited to, a glass capillary viscometer or a vibrating needle viscometer, a rheometer, a rotational rheometer, and the inclined plane test, but any suitable method can be utilized. When the pH is adjusted upwards to the raised pH the viscosity of the mixture increases, but does not achieve its viscosity prior to exposure to acidic conditions, revealing that a marked, irreversible, and permanent chemical change has occurred from the initial protein-containing mixture derived from the biomass.

Without wanting to be bound by any particular theory it is believed that subjecting the proto-protein to the delipidation and/or acid wash and/or other processes described herein may free or dissociate bound lipids by making (possibly irreversible) conformational changes in the proto-protein. It may also result in cleavage of covalently bound lipid-protein conjugates. The acid wash step does not truly hydrolyze the proteins in the biomass, but rather may free lipid moieties from the proteinacious (proto-protein) molecules in the biomass. The step may cause a conformational change in the proteins, and thereby free the lipidic moieties and allow them to be removed. It may also result in cleavage of covalently bound lipid-protein conjugates. These processes may make the lipid species (or other solvent soluble molecules) available for removal during solvent washing and/or extraction steps. These steps, and possibly in combination with the additional steps described herein, are believed to thus remove the portions of the proto-protein that give the undesirable organoleptic properties, and thus provide the organoleptically acceptable protein-containing material that is the food or food ingredient of nutritional interest in the invention, which is thus harvested. The protein-containing food or food product produced by the processes described herein is thus a markedly different molecule than the proto-protein that begins the processes.

Post-Acid Wash Re-Washing steps

Following the acid wash step there can be one or more steps of solvent washing, each optionally followed by a step of centrifugation to achieve a pellet, and resuspension in a solvent. The solvent can be any appropriate solvent as described herein for a solvent washing and/or delipidation step. After the one or more reworking or solvent washing steps (if performed) post acid wash, the protein mixture can be optionally dried in a rotary evaporator to make a protein concentrate, which can be utilized as a food or food ingredient.

### Pasteurization

In some embodiments the methods of producing a protein product include one or more steps of pasteurization, which can occur early in the production process. In one embodiment the pasteurization step(s) is performed prior to the acid wash step(s) (when performed). Thus, in one embodiment the methods involve performing one or more pasteurization step(s) on the biomass, which can be performed prior to performing one or more acid wash step(s) on the biomass. It has been discovered unexpectedly that by performing these steps in the recited order one is able to minimize the formation of lyso-phospholipids, free fatty acids, and secondary lipid oxidation products. Without wanting to be bound by any particular theory it is believed that the pasteurization step may destroy cellular lipases, which are therefore no longer available to break down fatty acids or other lipids in the mixture, which would then go on to become oxidized and form the compounds that give an unpleasant taste or smell and a protein with unacceptable organoleptic properties. These steps therefore produce a protein food ingredient that is substantially more pleasing in terms of taste and smell. The order of steps can include a step of pasteurization followed by a step of acid washing. In one embodiment the order of steps can be a step of pasteurization followed by a step of mechanical homogenization (e.g. bead milling), followed by a step of acid washing. Additional steps can be added or subtracted as disclosed herein.

In some embodiments a pasteurization step can involve raising the temperature of the biomass to at least 45° C. or at least 50° C. or at least 55° C. or about 60° C. or 60-65° C. or 63-68° C. or about 70° C. and holding it at said temperature for a period of time of at least 10 minutes or at least 15 minutes or at least 20 minutes or at least 25 minutes or about 30 minutes or 25-35 minutes or more than 35 minutes or 35-60 minutes or for more than 60 minutes.

### Proto-Protein

In some embodiments the biomass contains a proto-protein, which is a protein-containing molecule which also contains or is closely associated with a significant non-protein moiety, which can comprise a lipid moiety or moieties. The proto-protein can be the protein produced by the microbe in its natural faun, and before being treated according to the methods described herein. The proto-protein is close to its natural form and has undesirable or unfavorable organoleptic taste and smell properties and would score relatively low on the “degree of liking” scale or other method of evaluating organoleptic properties. Various algae and microbes produce proteins with these characteristics, and in some embodiments the proto-protein is an algal protein with undesirable organoleptic properties. In the methods of the invention the proto-protein is converted into the protein-containing food or food ingredient, which has more desirable or acceptable organoleptic properties and scores higher than the proto-protein on methods of evaluating such properties. Without wanting to be bound by any particular theory it is believed that the proto-protein may contain a lipidic component that gives the undesirable organoleptic taste and/or smell properties. Removal or disruption of this protein (or its lipidic components) can result in an improvement to acceptable or desirable organoleptic properties. In addition to (or instead of) lipid moieties the proto-protein can have other, molecular components or moieties that cause it to have (or worsen) its undesirable organoleptic properties. Therefore by applying the methods described herein the protein component of the biomass is converted into an organoleptically acceptable protein composition of the invention.

The molecular weight distribution of the proto-protein refers to the percentage of proto-protein molecules having a molecular weight within a specified size range or ranges. For example, the proto-protein may have a molecular weight distribution so that at least 50% or at least 60% or at least 70% of the proto-protein molecules (by weight) have a molecular weight of between about 10,000 and about 100,000 daltons, or from about 10,000 to about 50,000 daltons, or from about 20,000 to about 100,000 daltons, or from about 20,000 to about 80,000 daltons, or from about 20,000 to about 60,000 daltons, or from about 30,000 to about 50,000 daltons, or from about 30,000 to about 70,000 daltons, all non-aggregated. In other embodiments at least 70% or at least 80% of the proto-protein molecules have a molecular weight of between about 10,000 and about 100,000 daltons, or from about 20,000 to about 80,000 daltons, or from about 30,000 to about 50,000 daltons, or from about 30,000 to about 70,000 daltons, all non-aggregated. In other embodiments the molecular weight distribution of the proto-protein may be such that less than 25% or less than 10% or less than 5% of the proto-protein molecules have a molecular weight below about 20,000 daltons or below about 15,000 daltons or below about 10,000 daltons. In some embodiments the protein composition produced by the methods of the invention can have any of the molecular weight sizes and ranges described above or otherwise herein.

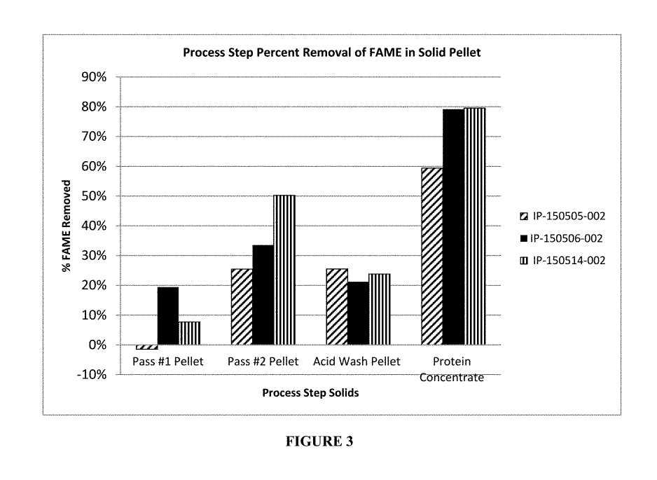

The methods of the invention convert a biomass containing a proto-protein into a proteinaceous or protein-rich concentrate. The fatty acid methyl ester (FAME) profile of the biomass at various steps can be evaluated to determine the quantity of lipidic material removed during the processes. Table 2 and FIG. 3 show the percent removal of FAME by the processing steps of the invention. Table 2—Percent removal of FAME by processing steps

| | Process Step | | | |
|---|---|---|---|---|
| | First Bead | Second Bead | | |
| Sample ID | Milling | Milling | Acid Wash | Final |
| 505-002 | — | 25% | 26% | 59% |
| 506-002 | 19% | 34% | 21% | 79% |
| 514-002 | 8% | 50% | 24% | 80% |
| average | 13.5% | 33% | 24% | |

The values in Table 2 reflect the percent of lipid removed by the indicated process step from the input material at that step. “Final” indicates the percent of total lipid removed versus the lipid content of the starting biomass. In various embodiments at least 60% or at least 70% or at least 75% of the lipid content in the fermented biomass that begins the methods is removed by the methods of the invention.

In some embodiments the biomass (or proto-protein) has a % FAME of greater than 9% or greater than 10% or greater than 11% or greater than 12% or greater than 13%. As a result of the methods described herein the % FAME can be reduced to less than 5% or less than 4% or less than 3% or less than 2% or less than 1% or less than 0.75% or less than 0.50%, all w/w.

The para-anisidine test (pAV), which is a standard test for secondary oxidation products of lipids, can also be used to monitor the amount of secondary oxidation products of lipids present after the processes of the invention, and therefore further characterize the protein product produced by the methods of the invention. In some embodiments the protein product produced by the methods of the invention has a pAV value of less than 2.0 or less than 1.0 or less than 0.9 or less than 0.8 or less than 0.7 or less than 0.6 or less than 0.5.

### More Methods

In some embodiments the invention provides methods of increasing the protein content of a biomass. In some embodiments the product of the invention is a protein-containing product having a higher protein concentration than the original biomass, with neutral color and improved organoleptic or hedonic properties. In various embodiments the protein-containing biomass that enters the processes of the invention can have a protein content of less than 65% or 50-65% or 40-70% or 45-65% or 45-70% (all w/w) and the protein content of the product protein composition of the methods is greater than 65% or greater than 68% or greater than 70% or greater than 72% or greater than 75% or greater than 77% or greater than 80% or 70-90% or 65-90% or 70-90% or 72-87% or 75-85% or 75-80%.

The invention also provides methods of lowering the arginine and glutamic acid (or glutamic acid and glutamine) content of a protein material. Arginine and glutamic acid (and glutamine) are two amino acids that are generously present in various types of food products. In many embodiments it is desirable to have a protein-rich food or food product that has a lower content of these common amino acids so that a more balanced supply of the 20 standard amino acids can be obtained in a food or food ingredient. It was discovered unexpectedly that the use of the defined medium produces a protein product with a lower amount of glutamic acid (or glutamic acid and glutamine) and/or arginine than in other protein compositions, and therefore is a nutritionally more balanced and better protein composition. In various embodiments the percent of glutamic acid (or glutamic acid and glutamine) is lowered from more than 21% or more than 22% to less than 20% or less than 18% or less than 16% or less than 15% or less than 14% or less than 13% or less than 12% (% of total amino acids). The percent of arginine can also be lowered from more than 9% to less than 9% or less than 8.5% or less than 8.0% or less than 7.5% or less than 7.0% (% of total amino acids). The methods of producing a protein composition with a lower arginine and/or glutamic acid (or glutamic acid and glutamine) content comprise any of the methods described herein.

### UCLAA

Amino, acid ratios (mg of an essential amino acid in 1.0 g of test protein/mg of the same amino acid in 1.0 g of reference protein) for 9 essential amino acids plus tyrosine and cysteine should be calculated by using the 1985 FAO/WHO/UNU suggested pattern of amino acid requirements for preschool children (2-5 years) (Joint FAO/WHO/UNU Expert Consultation. Energy & Protein Requirements. WHO Tech. Rept. Ser. No. 724. World Health Organization, Geneva Switzerland (1985)). This reference pattern, shown in FIG. 1, contains (mg/g protein): His, 19; Ileu, 28; Leu 66; Lys, 58; Met+Cys, 25; Phe+Tyr, 63; Thr, 34; Trp, 11; and Val 35. The lowest amino acid ratio is termed amino acid score. For example, a pinto bean sample contained 30.0, 42.5, 80.4, 69.0, 21.1, 90.5, 43.7, 8.8, and 50.1 mg/g protein of His, Ile, Leu, Lys, Met+Cys, Phe+Tyr, Thr, Trp, and Val, respectively. The respective amino acid (His, Ile, Leu, Lys, Met+Cys, Phe+Tyr, Thr, Trp, and Val) ratios for the bean sample would be 1.58, 1.52, 1.22, 1.19, 0.84, 1.44, 1.28, 0.80, and 1.43. This would then result in an uncorrected amino acid score of 0.80 with tryptophan as the first limiting amino acid.

### Protein Quality

All proteins are not equal since the quality of a protein and its absorption tendencies affect how much of the protein will actually be available to an organism consuming it. While UCLAA is a useful measure of protein value other measures are also useful for assessing protein quality. Protein Digestibility-Corrected Amino Acid Score (PDCAAS) is one method of evaluating protein quality based on both the amino acid requirements of humans and their ability to digest the protein. In various embodiments any of the protein compositions of the invention have a PDCAAS score of at least 0.60 or at least 0.62 or at least 0.65 or at least 0.67 or at least 0.70 or at least 0.72 or at least 0.75 or at least 0.77 or at least 0.80. Any of the protein compositions can also have an in vitro digestibility value of at least 0.86 or at least 0.88 or at least 0.90 or at least 0.92 or at least 0.94 or at least 0.95 or at least 0.96.

The Protein Efficiency Ratio (PER) and Biological Value (BV) are other measures of the quality of proteins. These are in vivo measures that have been closely correlated to PDCAAS which evaluates the extent to which a protein source is bio-available to the human or animal consumer. Higher scores of protein availability indicate the protein provides more of the essential amino acids, including the branched-chain amino acids that have a greater effect on protein synthesis. Another known method of evaluating protein quality is the in vitro method called Animal Safe Accurate Protein (ASAP) Quality method. This method has the advantage of being an in vitro method and eliminating animal studies. ASAP involves digestion with pepsin at pH 2, digestion with trypsin/chymotrypsin at pH 7.5, a TCA precipitate, reaction with ninhydrin, quantification by absorbance, and an adjustment of the result by amino acid composition. ASAP has also been closely correlated to the results obtained from a PDCAAS study in rats. The protein composition of the invention scores higher on any one or more of the named methods of evaluating protein quality. In various embodiments the protein composition of the invention has a ASAP score of at least 0.60 or at least 0.63 or at least 0.65 or at least 0.67 or at least 0.70 or at least 0.72 or at least 0.75 or at least 0.77 or at least 0.80.

While not necessarily, the protein compositions of the invention can be provided with an effective amount of an added preservative. The preservative can be any approved for use in food products for humans and/or animals.

### Calculation of UCLAA

The UCLAA is calculated by considering the mg of each of these amino acids per gram of protein and dividing it by the mg/g amino acid that is recommended for a 2-5 year old child by the Food and Agriculture Organization (FAO) of the United Nations (e.g. shown in Table 3) to obtain a UCLAA value for each of the amino acids. The lowest value calculated among the nine essential amino acids is the UCLAA score for the particular protein (although, as noted, phe+tyr can be measured together and met+cys can be measured together as part of the essential amino acids). The UCLAA score for the protein material of the invention can be at least 0.85, or at least 0.88, or at least 0.90, or at least 0.92 or at least 0.95, or at least 1.0, or at least 1.02, or at least 1.05, or at least 1.07, or at least 1.10. The protein material of the invention can also have a UCLAA score of greater than 1.0 for all of the essential amino acids. Table 3 shows UCLAA scores for a protein material prepared according to the invention and the UCLAA values achieved.

The invention in all its aspects is illustrated further in the following Examples. The Examples do not, however, limit the scope of the invention, which is defined by the appended claims.

### EXAMPLE 1

### Fermentation

This example illustrates a specific method for producing a dried protein material or concentrate (e.g., a powder) containing proteinaceous material from algal biomass. But persons of ordinary skill with resort to this disclosure will realize other embodiments of the methods, as well as that one or more of the steps included herein can be eliminated and/or repeated. Furthermore, any of the steps described herein can be included in any of the methods.

In this example algae (or chytrids) of the genus Aurantiochytrium sp. were used and were cultivated in a fermenter containing a defined medium as described above and in Table 1 containing glucose which supplied a source of organic carbon. The medium also contained macronutrients a trace minerals solution. The culture was maintained at 30° C. for 24 hours with 300-80 rpm of agitation, 0.1 vvm to 1.0 vvm aeration, 50% dissolved oxygen, and pH controlled to 6.3±0.1.

### EXAMPLE 2

### Post Fermentation Processing

100 kg of chytrid (Aurantiochytrium) fermentation broth (40 kg of solids at 50% protein) was harvested after fermentation and growth per Example 1. After centrifugation, the biomass was washed with aqueous solution followed by another centrifugation and the washed biomass was pasteurized at 65° C. for 15 seconds in a single pass HTST pasteurizer. Pasteurized biomass was then lysed and homogenized in a recirculating bead mill using 200-proof ethanol at a 1:1 (v/v) ethanol to solvent ratio to remove lipids and carbohydrates. The cells were lysed in the bead mill for 15 minutes at 35° C. using 1.0 mm beads, centrifuged to remove miscella and passed through again for an additional 15 minutes under the same conditions. The delipidated biomass was then centrifuged and the pellet was resuspended in water with antioxidants to undergo the acid washing step by lowering the pH to 3.5 for 30 minutes with H₂SO₄ and then raising the pH to 4.5 with NaOH for 1 hour. After pelleting the acid washed biomass was washed once with ethanol, centrifuged, and then passed through a high shear mixer twice for 15 minutes each with a centrifugation step after each mixing. Antioxidants were added to the pellet which underwent solvent extraction via high vacuum desolventization and then was converted into a dried protein concentrate by freeze drying.

### EXAMPLE 3

### Analysis

The dried protein concentrate (DPC) obtained from lots processed as described in Examples 1-2 were analyzed and found to have the amino acid composition as shown below in Table 3.

Table 3 below shows the UCLAA score for the dried protein concentrate (DPC) of the invention. The UCLAA was calculated as explained herein and it is shown each of the nine essential amino acids in humans is greater than or equal to 1.0, and therefore the UCLAA score for the protein composition is greater than 1.0. Table 3 also compares the dried protein concentrate of the invention to other commercial protein compositions such as whey, soy, and pea proteins, showing whey protein has a UCLAA score of 0.88, soy protein 0.93, and pea protein 0.73.

TABLE 3
Comparison of UCLAA Scores of Various Proteins

| | FAO | | | | | |
|---|---|---|---|---|---|---|
| | Recommended | | | | | |
| | Values | DPC from | | | | |
| | (2-5 yr old | defined | Whey | Soy | | DPC |
| | child) | media | Protein | Protein | Pea | from |
| ESSENTIAL | mg a.a. | UCLAA | Conc. | Conc. | Protein | rich |
| AMINO ACIDS | per g protein | score | (n = 2) | (n = 9) | Conc. | media |
| Histidine | 19 | 1.01 | 1.10 | 1.39 | 1.07 | 1.08 |
| Isoleucine | 28 | 1.43 | 2.07 | 1.61 | 1.40 | 1.16 |
| Leucine | 66 | 1.05 | 1.61 | 1.19 | 1.04 | 1.02 |
| Lysine | 58 | 1.12 | 1.63 | 1.10 | 1.04 | 1.10 |
| Methionine + | 25 | 1.43 | 1.61 | 0.93 | 0.64 | 1.49 |
| Cysteine | | | | | | |
| Phenylalanine + | 63 | 1.15 | 0.88 | 1.43 | 1.19 | 1.12 |
| Tyrosine | | | | | | |
| Threonine | 34 | 1.22 | 1.89 | 1.08 | 0.98 | 1.17 |
| Tryptophan | 11 | 1.15 | 1.68 | 1.33 | 0.78 | 0.73 |
| Valine | 35 | 1.36 | 1.62 | 1.33 | 1.18 | 1.49 |
| Essential Amino | 33.9% | 47.6% | 50% | 42.5% | 44% | 31.5% |
| Acids | | | | | | |
| % of total protein | | | | | | |
| Branched Chain | 12.9% | 18.6% | 19.7% | 18.0% | 18.3% | 12.3% |
| Amino Acids | | | | | | |
| % of total protein | | | | | | |
| Total Protein | | 77.4% | 82% | 65-72% | 82% | 66.6% |
| Content (N × 6.25) | | | | | | |

As shown in Table 3, histidine has the lowest UCLAA score at 1.01, and therefore the protein composition has a UCLAA score of 1.01. As also shown, while soy protein or pea protein have a higher UCLAA score for many amino acids, the score for met+cys is only 0.93 and 0.64, respectively. While whey protein also has a higher UCLAA score for several amino acids, its score for phe+tyr is only 0.88. Therefore the protein material prepared according to the invention is shown to provide a higher UCLAA score and a more balanced nutritional profile than other commercial proteins. The last column also compares the protein composition produced by fermentation in a defined medium from column 3 with the same biomass-produced protein composition produced in a rich medium. The rich medium is similar to the defined medium but also contains at least a trace amount of organic nitrogen. As shown, the protein composition from the rich medium has a UCLAA score of only 0.73.

### EXAMPLE 4

Table 4 below illustrates a comparison between the dried protein concentrate (DPC) prepared according to the invention using a defined fermentation medium according to Examples 1-2 or Table 1 versus various reference proteins such as egg, Spirulina, or Chlorella proteins. DPC values are shown as sum of the amino acids and as % of total protein based on Dumas, total N×6.25.

TABLE 4

| | | | | | DPC | |
|---|---|---|---|---|---|---|
| | | | | | (Dumas | |
| | | | Chlorella | DPC | protein) | |
| | Spirulina | Chlorella | protothecoides | (sum of | (total | |
| egg | platensis | vulgaris | CS41 | amino acids) | N × 6.25) | Amino Acid |
| 11 | 11.8 | 9 | 7.1 | 10.1 | 8.4 | Aspartic Acid |
| 5 | 6.2 | 4.8 | 4.9 | 4.9 | 4.1 | Threonine |
| 6.9 | 5.1 | 4.1 | 5.1 | 5 | 4.1 | Serine |
| 12.6 | 10.3 | 11.6 | 10.3 | 13.7 | 11.4 | Glutamic |
| | | | | | | Acid |
| 4.2 | 4.2 | 4.8 | 5.6 | 4.1 | 3.4 | Proline |
| 4.2 | 5.7 | 5.8 | 5.5 | 5.1 | 4.2 | Glycine |
| 2.4 | 9.5 | 7.9 | 6.2 | 6.9 | 5.7 | Alanine |
| 7.2 | 7.1 | 5.5 | 5.2 | 5.6 | 4.7 | Valine |
| 6.6 | 6.7 | 3.8 | 3.7 | 4.7 | 3.9 | Isoleucine |
| 8.8 | 9.8 | 8.8 | 5.6 | 8.3 | 6.9 | Leucine |
| 4.2 | 5.3 | 3.4 | 4.7 | 3.8 | 3.2 | Tyrosine |
| 5.8 | 5.3 | 5 | 5.5 | 4.9 | 4.0 | Phenylalanine |
| 5.3 | 4.8 | 8.4 | 4.9 | 7.7 | 6.4 | Lysine |
| 2.4 | 2.2 | 2 | 3 | 2.2 | 1.8 | Histidine |
| 6.2 | 7.3 | 6.4 | 13.4 | 7.6 | 6.4 | Arginine |
| 2.3 | 0.9 | 1.4 | 1.6 | 1.3 | 1.1 | Cystine |
| 3.2 | 2.5 | 2.2 | 2.1 | 2.9 | 2.4 | Methionine |
| 1.7 | 0.3 | 2.1 | 0.49 | 1.4 | 1.2 | Tryptophan |
| 100 | 105 | 97 | 94.89 | 100 | 83.3 | Total |

Table 4 shows that each of the comparison proteins are deficient in some significant way. Egg and Spirulina are deficient in lysine, Spirulina is also deficient in tryptophan, and Chlorella is deficient in methionine. The algal protein concentrate of the invention provides a more nutritionally balanced protein composition and therefore a better quality food as evidenced by the UCLAA score and other nutritional parameters.

Table 5 below shows how the total amino acid composition in the final protein composition changes as a result of using a rich medium (containing organic nitrogen) versus a defined medium that lacks organic nitrogen in the fermentation process. Note that the protein composition produced in the defined medium contains more than 50% less glutamic acid, more than 30% less arginine and more than 10% less cystine.

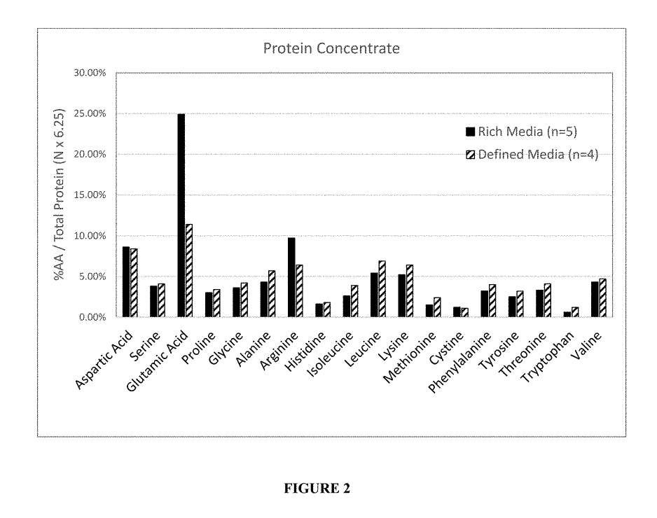

Table 5 below also illustrates that a protein composition of the invention prepared from biomass growing on a defined medium of Example 1 produces at least 5% or at least 6% or at least 7% or at least 8% more of each essential amino acid versus growth on a rich medium, and also produces at least 15% or at least 18% or at least 20% or at least 22% or at least 24% more essential amino acids versus the same biomass grown on a rich medium. The protein composition also contains at least 25% or at least 30% or at least 35% or at least 40% or at least 45% or at least 50% more branched chain amino acids versus the same biomass grown on a rich medium. This is graphically depicted in FIG. 2. Notably, at least 90% or at least 95% or about 100% more tryptophan was produced, which is often a challenging amino acid to find in usual dietary sources. At least 50% or at least 55% or at least 57% or at least 59% more methionine was produced in the defined versus rich medium. At least 45% or at least 47% or at least 49% more isoleucine was produced versus the rich medium. At least 18% or at least 20% or at least 22% or at least 24% more phenylalanine was produced versus the rich medium.

TABLE 5
Comparison of amino acid composition on defined medium versus rich medium for a defined
protein concentrate from Aurantiochytrium (values as a % of total protein based
on Dumas total N × 6.25; *indicates an essential amino acid for humans)

| | | | | % change in content |
|---|---|---|---|---|
| | Rich medium | Defined medium | Absolute | when switching from |
| | average | average | difference | rich to defined medium |
| Aspartic Acid | 8.6% | 8.4% | 0.2% | (2%) |
| Serine | 3.8% | 4.1% | 0.3% | 8% |
| Glutamic acid | 24.9% | 11.4% | 13.5% | (54%) |
| Proline | 3.0% | 3.4% | 0.4% | 12% |
| Glycine | 3.6% | 4.2% | 0.6% | 17% |
| Alanine | 4.3% | 5.7% | 1.4% | 33% |
| Arginine | 9.7% | 6.4% | 3.3% | (34%) |
| Histidine* | 1.6% | 1.8% | 0.2% | 12% |
| Isoleucine* | 2.6% | 3.9% | 1.3% | 50% |
| Leucine* | 5.4% | 6.9% | 1.5% | 28% |
| Lysine* | 5.2% | 6.4% | 1.23% | 24% |
| Methionine* | 1.5% | 2.4% | 0.9% | 60% |
| Cystine | 1.2% | 1.1% | −0.14% | (12%) |
| Phenylalanine* | 3.2% | 4.0% | 0.8% | 25% |
| Tyrosine | 2.5% | 3.2% | 0.67% | 26% |
| Threonine* | 3.3% | 4.1% | 0.8% | 24% |
| Tryptophan* | 0.6% | 1.2% | 0.6% | 100% |
| Valine* | 4.3% | 4.7% | 0.4% | 9% |
| Essential Amino | 31.5% | 39.7% | 8.2% | 26% |
| Acids | | | | |
| % of total protein | | | | |
| Branched Chain | 12.3% | 15.5% | 6.5% | 53% |
| Amino Acids | | | | |
| % of total protein | | | | |

It is therefore seen that each of the essential amino acids increased by at least 8% or by 9% or more when the biomass was fermented in a defined medium versus a rich medium. Furthermore, the protein composition of the invention also contained significantly higher amounts of branched chain amino acids. This is also graphically depicted in FIG. 2.

Of the essential amino acids valine was more than 7% or more than 8% or more than 9% higher, histidine was more than 10% or more than 12% higher, isoleucine more than 45% or more than 48% higher, leucine more than 25% higher, methionine more than 55% or more than 58% higher, phenylalanine more than 23% higher, threonine more than 20% higher, and tryptophan more than 90% or more than 95% higher.

### EXAMPLE 5

This example provides a general scheme for producing a dried protein material or concentrate (e.g., a powder) from algal biomass. This example illustrates a specific method but persons of ordinary skill with resort to this disclosure will realize other embodiments of the methods, as well as that one or more of the steps included herein can be eliminated and/or repeated. Furthermore, any of the steps described herein can be included in any of the methods.

In this example algae (chytrids) of the genus Aurantiochytrium sp. were used and were cultivated in a fermenter containing a rich medium containing 0.1 M glucose and 10 g/L of yeast extract, which supplied a source of organic carbon. The medium also contained macronutrients and a trace mineral solution. The culture was maintained at 30° C. for 24 hours with 300-80 rpm of agitation, 0.1 vvm to 1.0 vvm aeration, 50% dissolved oxygen, and pH controlled to 6.3±0.1.

After harvesting, the fermentation broth was removed from the cells via centrifugation and the resulting biomass pellet was diluted in water and re-centrifuged (cell wash). The resulting paste was mixed with antioxidants to prevent oxidation of oils and other components, and then drum dried to remove water, which produced a dry cellular material.

A pasteurization step was performed by raising the temperature of the broth to about 65° C. and holding it at that temperature for about 30 minutes. The dry cells were then thoroughly lysed in 100% ethanol in a bead mill. This is a homogenization and solvent extraction step and removes soluble substances such as lipids, and the delipidated biomass is separated from the miscella using centrifugation.

The biomass was then subjected to an acid wash via titration of 1 N H₂SO₄, until the pH was acidified to about 3.5. The biomass was then mixed for about 30 minutes. The pH was then raised to about 4.5 with 1 N NaOH and the biomass mixed for 1 hour.

The acid washed material was then centrifuged and the supernatant removed. The pellet was then subjected to two re-washing/extraction steps, which involved two rounds of suspension in 100% ethanol followed by high shear mixing and centrifugation. The supernatant was decanted to maximize extraction and removal of undesired compounds. The high shear mixing was performed with a rotor stator type mixer (e.g., IKA ULTRA-TURRAX®) with the temperature being controlled at <20° C. by an ice bath. The resultant ethanol-washed pellet (biomass) was then dried by placing in a modified rotary evaporation flask to promote tumble-drying at room temperature under moderate vacuum. After approximately 4 hours the material changed from a paste to a powder. At this point, the material was removed from the rotary evaporator and ground to a fine powder with a mortar and pestle. This material was then placed on an aluminum tray in a vacuum oven at 90° C. for approximately 11 hours to remove any residual solvent or moisture. Once dry, the material was passed through a particle size classifier to remove particles greater than 300 um in size. These particles can be completely removed from the final product if desired, or further ground up and returned back to the final product. The end result of the process was a uniform, neutral colored powder of neutral hedonic character, which can be packaged under nitrogen and stored in a −80° C. freezer.

### EXAMPLE 6

Three independent fermentations were performed on algae of the genus Aurantiochyrium sp. in medium similar to that of Example 5 and the mass of the acid wash supernatant stream was quantitated, and protein determined by the Dumas method (quantitative determination of Nitrogen by elemental analysis). As shown in Table 6 below, the acid wash removed between 8.8% and 15.8% of the initial feedstock mass. Converting nitrogen content to protein content by the calculation (N*6.25) estimates the protein content of the acid wash solids is 12.15% to 15.50% protein. The protein removed by the acid wash step ranged from 2.01% to 3.4% of the initial protein in the feed.

TABLE 6
Acid Wash Supernatant Masses and Protein

| | Sample 825 | Sample 908 | Sample 319 |
|---|---|---|---|
| Mass | 15.80% | 14.00% | 8.80% |
| removed, % | | | |
| of feed | | | |
| Acid wash | 12.60% | 12.15% | 15.50% |
| Solids % | | | |
| protein | | | |
| Protein, % of | 3.40% | 2.70% | 2.01% |
| feed Protein | | | |

### EXAMPLE 7

An additional example of the impact of the acid wash upon amino acid composition is shown below. Two separate processes were performed where the acid wash supernatant was dialyzed and dried, and analyzed for amino acid composition. An Aurantiochytrium (chytrid) strain was processed as described above, the acid wash supernatant and algal protein concentrate were analyzed and compared to the initial dry biomass feed. It was found that glutamic acid (or glutamic acid and glutamine) and arginine are selectively removed from the biomass during the acid wash.

Without wanting to be bound by any particular theory it is believed that the acid wash step prepares the proteinaceous material for a preferential protein removal so that the content of generally unwanted amino acids arginine, glutamic acid (or glutamic acid and glutamine), and hydroxyproline is lowered in the final protein product versus the raw algal protein. After acid washing the samples were subjected to two additional rounds of solvent washing. It is also believed that the acid wash step exposes or otherwise renders certain proteins in the proteinaceous material susceptible to removal, and these removed proteins are high in the content of these unwanted amino acids. This is advantageous since it allows for the production of a more nutritionally balance protein material. The content of arginine and glutamic acid (or glutamic acid and glutamine) and hydroxyproline is measured by calculating the ratio of each amino acid in the final protein product pellet versus the content in the supernatant. Thus a low ratio indicates the amino acid is more prevalent in the supernatant. Table 6 below illustrates the data and shows that the ratio for these three amino acids is less than 2 or less than 1 or less than 0.75 for arginine, less than 2 or less than 1 or less than 0.75 or less than 0.60 for glutamic acid (or glutamic acid and glutamine), and less than 2 or less than 1 or less than 0.75 or less than 0.55 for hydroxyproline.

TABLE 7

| | Acid Wash | Final | Ratio of Pellet |
|---|---|---|---|
| Amino Acid % | Supernatant | Product in | to AWS |
| of sample | (AWS) | Pellet | amino acid composition |
| Methionine | 0.08% | 0.83% | 10.35 |
| Cystine | 0.13% | 0.48% | 3.80 |
| Lysine | 0.76% | 4.38% | 5.76 |
| Phenylalanine | 0.01% | 2.82% | 315.04 |
| Leucine | 0.21% | 4.56% | 21.26 |
| Isoleucine | 0.19% | 2.33% | 12.40 |
| Threonine | 0.50% | 3.07% | 6.13 |
| Valine | 0.33% | 3.66% | 11.07 |
| Histidine | 0.35% | 1.76% | 5.04 |
| Arginine | 15.61% | 11.12% | 0.71 |
| Glycine | 0.95% | 3.23% | 3.40 |
| Aspartic Acid | 1.17% | 6.86% | 5.86 |
| Serine | 0.57% | 3.27% | 5.71 |
| Glutamic Acid | 76.24% | 41.97% | 0.55 |
| Proline | 0.35% | 2.64% | 7.58 |
| Hydroxyproline | 0.05% | 0.03% | 0.49 |
| Alanine | 1.70% | 4.20% | 2.48 |
| Tyrosine | 0.72% | 2.27% | 3.18 |
| Tryptophan | 0.09% | 0.79% | 8.87 |
| TOTAL: | 100.00% | 100.00% | 1.00 |

### EXAMPLE 8

### Lipid Removal During Acid Wash

Two processes using the same biomass source (chytrid #705) were performed to show the effect of the acid wash on FAME content in the protein concentrate. After drum drying the initial biomass from the fermenter the samples were subjected to two rounds of mechanical homogenization by bead milling followed by a step of solvent washing in 100% isopropyl alcohol. Sample 225-002/A was subjected to an acid washing step as describe in Example 1 while sample 225-002/A.2 was not. Each sample was then subjected to two reworking solvent washing steps in 100% isopropyl alcohol before being dried in a rotary evaporator. The results clearly show the lowering of the final FAME content in the protein product from 2.19% of final dry weight to 0.89% of final dry weight, which can be attributable to the acid washing step.

TABLE 8

| | | | | Protein |
|---|---|---|---|---|
| | | | | concentrate |
| | Experimental | | % Protein | FAME % of |
| Lot Designation | Descriptor | Sample Descriptor | (Dumas) | dry weight |
| 225-002/A | Acid Washed | Drum Dry/Iso-propyl alcohol | 83.66% | 0.89% |
| | (AW) | mill/AW/Rework/Drying | | |
| 225-002/A.2 | Non-Acid | Drum Dry/IPA Mill/Rework/ | 81.22% | 2.19% |
| | Washed | Drying (No acid wash) | | |

The stepwise efficiency of removing available lipids through the process was examined in order to see the specific contribution of the acid wash step for the removal of lipids. FIG. 3 shows the results for three independent treatments performed using the strain from Example 7. Ethanol was used as the solvent prior to and after the acid wash. The acid wash step included a first adjustment to pH 3.5 with 1 N H₂SO₄ per Example 5, followed by adjustment to pH 4.5 with 1 N KOH. For each significant process step, the resultant solids were analyzed for FAME content. The acid wash step removed 26%, 21%, and 24% of the lipid present in the biomass after the bead mill processing (samples 505-002, 506-002, and 514-002, respectively). The data show that when an acid wash step is included in the preparation method the percent of FAME in the protein produced was reduced to 0.89%, or to less than 1%. When the acid wash step is omitted from the process the percent FAME in the protein produced was 2.19%, or higher than 2%.

### EXAMPLE 9

The para-anisidine test (pAV), which is a standard test for secondary oxidation products of lipids, was used to monitor the amount of secondary oxidation products of lipids present after certain steps of the methods. The pAV values were determined for four independently-fermented batches of chytrid biomass, tested at three steps in the downstream processing: water-washed biomass collected immediately at the conclusion of fermentation (washed pellet); pasteurized biomass; final protein concentrate (after acid washing and two re-working steps). The downstream process steps are described in Table 9 below.

TABLE 9
pAV Relative to Soy Protein

| p-AV relative to | | Pasteurized | Protein |
|---|---|---|---|
| soy protein | Washed Pellet | Biomass | Concentrate |
| IP-150505-002 | 4.0 | 4.0 | 0.8 |
| IP-150506-002 | 3.6 | 5.4 | 0.5 |
| IP-150511-002 | 3.5 | 2.5 | 0.8 |
| IP-150514-002 | 1.6 | 1.5 | 0.4 |

The values shown in Table 9 are ratios of the pAV of the algal protein concentrate relative to the pAV value determined, for a commercially available protein isolate produced from soybean (which is used as a benchmark standard). The data show that prior to the processing steps of bead milling/ethanol extraction and acid washing, the algal protein concentrate has a higher content of secondary lipid oxidation products than does a soybean protein isolate. But after two bead milling/ethanol solvent washing steps and one acid washing step with two reworking solvent washing steps, each of the four samples of protein product have a lower content of secondary lipid oxidation products than the soybean protein isolate. Thus, the steps of the invention, including the acid washing, improve the quality of the protein concentrate with respect to lipid content (and therefore lipid oxidation) and organoleptic properties.

### EXAMPLE 10

This example shows the robustness of the methods as applied to other microbial species. Table 10 compares the production of a DPC using a defined medium versus a rich medium for both a yeast and an algae. It is seen that in both the yeast and the algae the UCLAA score increases substantially in the defined medium.

TABLE 10

| | FAO | | | | |
|---|---|---|---|---|---|
| | Recommended | | | | |
| | Values | | | | |
| ESSENTIAL | (2-5 yr old | Kluyveromyces | Kluyveromyces | Chlorella | Chlorella |
| AMINO | child) mg a.a. | Whole biomass | Whole biomass | Whole biomass | Whole biomass |
| ACIDS | per g protein | rich medium | defined medium | rich medium | defined medium |
| Histidine | 19 | 0.96 | 1.10 | 0.62 | 0.68 |
| Isoleucine | 28 | 1.76 | 1.94 | 0.78 | 0.91 |
| Leucine | 66 | 1.19 | 1.32 | 0.70 | 0.77 |
| Lysine | 58 | 1.41 | 1.51 | 0.54 | 0.68 |
| Methionine + | 25 | 1.32 | 1.12 | 0.65 | 0.84 |
| Cysteine | | | | | |
| Phenylalanine + | 63 | 1.36 | 1.48 | 0.67 | 0.76 |
| Tyrosine | | | | | |
| Threonine | 34 | 1.49 | 1.59 | 0.85 | 0.86 |
| Tryptophan | 11 | 1.25 | 1.19 | 0.83 | 0.94 |
| Valine | 35 | 1.84 | 1.78 | 0.96 | 1.04 |
| Essential | 33.9% | 47.5% | 50.0% | 39.2% | 45.0% |
| Amino Acids | | | | | |
| % of total | | | | | |
| protein | | | | | |
| Branched | 12.9% | 19.2% | 20.3% | 24.1% | 27.4% |
| Chain Amino | | | | | |
| Acids | | | | | |
| % of total | | | | | |
| protein | | | | | |
| Total Protein | | 65.0% | 51.0% | 10.2% | 11.3% |
| Content (N × | | | | | |
| 6.25) | | | | | |

### EXAMPLE 11

### Sensory Panel

Reports from sensory panels composed of persons selected to evaluate the organoleptic properties of the protein composition have demonstrated the processes of the present invention result in a protein composition having improved and acceptable organoleptic (hedonic) properties compared to unprocessed product.

A powdered protein composition (DPC) prepared according to the methods described herein was mixed with water and given in blind taste and smell tests to multiple panels of 3-5 persons using the “sip and spit” method and compared with a soy standard. All persons on all panels rated the protein composition of the invention as “organoleptically acceptable.” Comments from the panels included that the fishy or briny taste and smell of unprocessed algal protein was hardly noticeable. Thus, the presence of an unpleasant fishy odor or taste, or ammonia-like odor or taste, or briny odor or taste was markedly decreased as a result of the process while the protein material maintained a high protein content.

### EXAMPLE 11A

### Sensory Panels

Persons of ordinary skill in the art understand how to assemble a sensory evaluation panel and evaluate food samples in a reliable manner, for example the 9 point hedonic scale, which is also known as the “degree of liking” scale can be utilized. (Peryam and Girardot, N. F., Food Engineering, 24, 58-61, 194 (1952); Jones et al. Food Research, 20, 512-520 (1955)). This example therefore provides only one scientifically valid manner of performing such evaluation.

A panel of six adult subjects (3 male and 3 female) evaluate the organoleptic taste and smell properties of eight protein products derived from algal (chytrid) biomass processed as described in Examples 1-2 (although a protein produced according to Example 5 will yield similar results). The subjects are randomly assigned an identifying letter A-F. Four of the eight samples are prepared according to the procedure of Examples 1-2, which includes one acid wash procedure (“test” samples). The other four samples are control samples, which have been prepared identically except they were not subjected to the acid washing step (“control” samples). After the samples are dried and obtained in powdered form, 1 gram of protein powder is dissolved in deionized water to make a 10% solution in a plastic tube. The eight samples are provided to each subject in random order and without any subject knowing the identity of any sample.

The samples are evaluated for whether the samples are organoleptically pleasing or unpleasant. The subjects are asked to consider the categories “fishy taste and/or smell” and “ammonia-like taste and/or smell” and “briny taste and/or smell” according to the following five point scale: 0—none; 1—slight; 2—moderate; 3—high; and 4—extreme. The subjects also evaluate the general organoleptic properties as acceptable or unacceptable, using soy protein similarly prepared as a standard, and whether the samples have organoleptic properties equal to, better, or worse than the soy protein sample. The subjects are instructed to assign the sample the lowest rating received in either category. The manner of testing is first to evaluate the aroma of the sample. If the subject rates the aroma a 3 or 4 in any category the sample is considered organoleptically unpleasant or unacceptable and no tasting is required. If the aroma rates between 0 and 2 the subject further tests the sample by the known “sip and spit” method, with sample being held in the mouth for 1-2 seconds.

In the aroma evaluation portion of the study, 5 of the 6 panel members rate all four control samples a 3, i.e., high fishy smell and/or high ammonia-like smell and/or high briny smell, and therefore organoleptically unacceptable. The subjects also rate the control samples as less pleasing than the soy protein sample. Therefore these 5 subjects do not proceed to the taste portion of the study for these samples and the samples are rated as having unpleasant or unacceptable organoleptic properties. The remaining subject rates three of the four control samples a “3”, and the remaining control sample a “2.” For the fourth control sample this subject proceeds to the taste portion and rates the remaining control sample a 3 and rates all samples less pleasing than the soy sample.

For the four test samples in the aroma portion of the study, 5 of the 6 subjects rate all four of the samples a “0” and equal to soy. The remaining subject rates three samples a “0” and equal to soy and one sample a 1 and less pleasing than soy.

The subjects then proceed to the taste portion of the study. For the taste portion five of the subjects rate all four samples a “0” for taste and equal to soy. The remaining subject rates three samples a “0” and equal to soy, and one sample a “1” and less pleasing than soy.

The data are summarized in Table 11 and show that the protein composition prepared according to the present invention has improved organoleptic properties versus samples prepared according to traditional methods. It is also seen that samples prepared according to the invention are clearly more likely to be equal to soy protein standard in organoleptic taste and smell properties and to have acceptable or desirable organoleptic properties.

TABLE 11
Samples Evaluated as either organoleptically pleasing or unpleasant

| | A | B | C | D | E | F |
|---|---|---|---|---|---|---|
| 1 test | S-0 | S-0 | S-0 | S-0 | S-0 | S-0 |
| | T-0 | T-0 | T-0 | T-0 | T-0 | T-0 |
| 2 test | S-0 | S-0 | S-0 | S-1 | S-0 | S-0 |
| | T-0 | T-0 | T-0 | T-1 | T-0 | T-0 |
| 3 test | S-0 | S-0 | S-0 | S-0 | S-0 | S-0 |
| | T-0 | T-0 | T-0 | T-0 | T-0 | T-0 |
| 4 test | S-0 | S-0 | S-0 | S-0 | S-0 | S-0 |
| | T-0 | T-0 | T-0 | T-0 | T-0 | T-0 |
| 5 control | S-3 | S-3 | S-3 | S-3 | S-3 | S-3 |
| 6 control | S-3 | S-2 | S-3 | S-3 | S-3 | S-3 |
| | | T-3 | | | | |
| 7 control | S-3 | S-3 | S-3 | S-3 | S-3 | S-3 |
| 8 control | S-3 | S-3 | S-3 | S-3 | S-3 | S-3 |

Although the disclosure has been described with reference to the above examples, it will be understood that modifications and variations are encompassed within the spirit and scope of the disclosure. Accordingly, the disclosure is limited only by the following claims.

1. A protein composition derived from cellular biomass and having an uncorrected limiting amino acid score of 0.88 or greater for all essential amino acids.

2. The protein composition of claim 1 wherein the biomass is derived from an algae.

3. The protein composition of claim 2 wherein the algae is a heterotrophic algae.

4. The protein composition of claim 1 having an uncorrected limiting amino acid score of greater than 0.94 for all essential amino acids.

5. The protein composition of claim 4 having an uncorrected limiting amino acid score of greater than 1.0 for all essential amino acids.

6. The protein composition of claim 1 comprising phe in an amount of 3.5% of total protein or greater, and tyr in an amount of 2.75% of total protein or greater.

7. The protein composition of claim 3 wherein the protein content is greater than 65%.

8. The protein composition of claim 7 wherein the lipid content is less than 10%

9. The protein composition of claim 8 wherein the lipid content is less than 2%.

10. The protein composition of claim 9 wherein the ash content is less than 8%.

11. The protein composition of claim 1 wherein the content of essential amino acids is greater than 35% of total protein.

12. The protein composition of claim 1 wherein the content of branched chain amino acids is greater than 16% of total protein.

13. The protein composition of claim 1 comprising:
a) leucine in an amount greater than 5.5% of total protein;
b) isoleucine in an amount greater than 3.0% of total protein;
c) glutamic acid in an amount less than 20% of total protein;
d) lysine in an amount greater than 5.5% of total protein; and
e) valine in an amount greater than 4.5% of total protein.

14. The protein composition of claim 13 comprising:
a) leucine in an amount greater than 6% of total protein;
b) lysine in an amount greater than 6% of total protein; and
c) glutamic acid in an amount less than 15% of total protein.

15. The protein composition of claim 3 wherein the composition has organoleptic taste and smell properties acceptable to a human.

16. The protein composition of claim 15 wherein the protein composition has organoleptic taste and smell properties at least equivalent to soy.

17. The protein composition of claim 3 wherein the heterotrophic algae is from the class Labyrinthulomycetes.

18. The protein composition of claim 17 wherein the composition is derived from a single source.

19. The protein composition of claim 17 that does not contain human allergens from peanut, milk, soy, nut, egg, whey, wheat, fish, shellfish, or pea at or above the lowest observed adverse effect level for the particular human allergen.

20. The protein composition of claim 17 wherein the Labyrinthulomycete is selected from the group consisting of: Thraustochytrium, Aurantiochytrium, and Schizochytrium.

21. The protein composition of claim 18 wherein the Labyrinthulomycete is Aurantiochytrium.

22. A method of producing a protein composition comprising:
a) cultivating a cellular biomass in a defined medium;
b) delipidating the biomass;
c) exposing the delipidated biomass to acidic conditions by adjusting the pH of the biomass to a depressed pH of less than 4.5 and holding the pH of the biomass at said depressed pH for at least 10 minutes; and
d) harvesting a protein composition comprising a UCLAA score of at least 0.88.

23. The method of claim 22 wherein exposing the delipidated biomass to acidic conditions comprises exposing the biomass to a pH of about 3.5 and the pH is held for about 30 minutes.

24. The method of claim 22 wherein the cellular biomass is algal biomass.

25. The method of claim 24 wherein the algal biomass is derived from an organism of the class Labyrinthulomycetes.

26. The method of claim 22 wherein the protein composition has organoleptic taste and smell properties acceptable to a human.

27. The method of claim 25 wherein the protein composition contains at least 75% protein w/w and less than 5% lipid content wlw.

28. A protein composition derived from cellular biomass and having organoleptic properties acceptable to a human.

29. The protein composition of claim 28 derived from cellular biomass and having acceptable organoleptic properties to a human.

30. The protein composition of claim 29 having an uncorrected limiting amino acid score of 0.88 or greater for all essential amino acids.
